(19) United States
(12) Patent Application Publication
(10) Pub. No.: US 2018/0193391 A1
(43) Pub. Date: Jul. 12, 2018

(54) INDUCTION OF LAMINA PROPRIA REGULATORY T CELLS

(71) Applicant: President and Fellows of Harvard College, Cambridge, MA (US)

(72) Inventors: Esen Sefik, Brookline, MA (US); Naama Geva-Zatorsky, Brookline, MA (US); Christophe Genoist, Brookline, MA (US); Dennis Kasper, Brookline, MA (US); Diane Mathis, Brookline, MA (US)

(21) Appl. No.: 15/739,438

(22) PCT Filed: Jun. 21, 2016
(86) PCT No.: PCT/US16/38502

§ 371 Date: Dec. 22, 2017

Related U.S. Application Data
(60) Provisional application No. 62/183,021, filed on Jun. 22, 2015.
(60) Provisional application No. 62/183,019, filed on Jun. 22, 2015.

(51) Int. Cl. A61K 35/745; A61P 1/04; A61K 35/747; A61K 9/00

(52) U.S. Cl. CPC A61K 35/745; A61P 1/04; A61K 35/747; A61K 31/575; A61K 9/0053; A61K 2300/00; A61K 9/0031

(57) ABSTRACT

Described herein are methods and compositions for the induction of the production of regulatory T cells and for the treatment and/or prevention of diseases associated with pathological immune responses, such as inflammatory bowel diseases.

### RELATED APPLICATIONS

This application claims the benefit of priority to U.S. Provisional Patent Application Ser. No. 62/183,019, filed Jun. 22, 2015, and U.S. Provisional Patent Application Ser. No. 62/183,021, filed Jun. 22, 2015, each of which are hereby incorporated by reference in their entirety.

### GOVERNMENT INTEREST

This invention was made with Government support under National Institutes of Health Grant R01-AI51530 and R56AI110630. The Government has certain rights in the invention.

### BACKGROUND

Regulatory T cells (Tregs) are a subset of T cells that help keep the immune system in check by suppressing immune responses, thereby reducing immune-mediated pathology, such as autoimmune disease, inflammatory disease and allergies. Tregs regulate the immune response through a number of mechanisms, such as the secretion of inhibitory cytokines, such as IL-10 and TGF-β, the expression of inhibitory receptor CTLA-4 and granzyme-mediated cytolysis.

One of the key regulators of Treg development and function is the transcription factor FoxP3. Reduced numbers of FoxP3 expressing Tregs are associated with autoimmune diseases such as systemic lupus erythematosus (SLE). Mutation of FoxP3 in humans results IPEX (immunodysregulation polyendocrinopathy enteropathy X-linked) syndrome, which is characterized by a dysfunction of regulatory T cells and subsequent autoimmunity.

Beyond their role in regulating immune activity, Tregs located in parenchymal tissues, referred to as “tissue Tregs,” are also involved in certain other, non-immunological, processes. For example, tissue Tregs that reside in visceral adipose tissue regulate metabolic parameters, while those located in muscle tissue channel tissue inflammation and regeneration following muscle injury.

An important population of tissue Tregs resides in the lamina propria (LP) of the digestive tract, and in particular in the colon. In fact, under normal conditions, compared with all other tissues, the intestinal lamina propria has the greatest population of Tregs. Such LP Tregs modulate responses to commensal microbes, thereby reducing the risk of intestinal immunopathologies. LP Tregs are an unusual population, which has provoked some contradictory observations. T cell receptors expressed by LP Tregs show a marked reactivity against microbial antigens, which are important drivers of LP Treg differentiation and/or expansion (Lathrop et al., Nature 478:250 (2011); Cebula et al., Nature 497:258 (2013)), and some LP Tregs may arise by conversion from FoxP3⁻ conventional T cells (Tconv) (Lathrop et al., Nature 478:250 (2011)), although arguments for a thymic origin have also been made (Cebula et al., Nature 497:258 (2013)). Many LP Tregs express marker profiles (Nrp1⁻, Helios⁻) that differ from Tregs found in peripheral lymphoid tissue (Bilate and Lafaille, Annu. Rev. Immunol. 30:733 (2012), though the significance of the different marker expression patterns is unclear. Generally, germ-free (GF) mice exhibit a reduced abundance of LP Tregs (Ai et al., Immunol. Rev. 259:60 (2014)), and colonization of GF mice by pools of microbes (particularly Schadler's flora and Clostridia combinations) elicited the differentiation or expansion of Helios⁻Nrp1⁻ LP Tregs (Geuking et al., Immunity 34:794 (2011); Atarashi et al., Nature 500:232 (2013); Atarashi et al., Science 331:337 (2011)). The ability of single microbes to induce colonic Tregs has been more controversial, and the need for complex combinations has been suggested Atarashi et al., Nature 500:232 (2013); Atarashi et al., Science 331:337 (2011); Faith et al., Sci. Transl. Med. 6:220ra11 (2014). Accordingly, induction of LP Tregs is a promising treatment for intestinal immunopathologies, including inflammatory bowel diseases such as ulcerative colitis and Crohn's disease.

### SUMMARY

In certain aspects, provided herein are methods and compositions for inducing the production of Rorγ⁺Helios⁻ lamina propria regulatory T cells (LP Tregs) and/or for treating or preventing a disease associated with a pathological immune response (e.g., an inflammatory bowel disease) in a subject.

In certain aspects provided herein is a method for inducing the production of Rorγ⁺Helios⁻ LP Tregs in a subject comprising administering to the subject a composition comprising a bacteria or a combination of bacteria that induce the production of Rorγ⁺Helios⁻ LP Tregs. In some embodiments, the composition further comprises a pharmaceutically acceptable carrier. In some embodiments, the composition is a food product supplemented with the bacteria. In some embodiments, the food product is or comprises a dairy product (e.g., yogurt, frozen yogurt, ice cream, milk or cheese). In some embodiments, the food product is a non-dairy food product. In some embodiments, the food product is a beverage. In some embodiments, the composition is administered orally. In some embodiments, the composition is administered rectally.

In certain aspects provided herein is a method of treating or preventing an inflammatory bowel disease in a subject comprising administering to the subject a composition comprising a bacteria or a combination of bacteria that induce the production of Rorγ⁺Helios⁻ LP Tregs. In some embodiments, the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease. In some embodiments, the disease is Crohn's disease or ulcerative colitis. In some embodiments, the composition further comprises a pharmaceutically acceptable carrier. In some embodiments, the composition is a food product supplemented with the bacteria. In some embodiments, the food product is or comprises a dairy product (e.g., yogurt, frozen yogurt, ice cream, milk or cheese). In some embodiments, the food product is a non-dairy food product. In some embodiments, the food product is a beverage. In some embodiments, the composition is administered orally. In some embodiments, the composition is administered rectally.

In some embodiments of the methods described herein, the subject has or is predisposed to a disease associated with a pathological immune response. In some embodiments, the disease is an inflammatory bowel disease (e.g., Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease). In some embodiments, the disease is Crohn's disease or ulcerative colitis.

In some embodiments of the methods described herein, the subject has reduced levels of bacteria present in their gut. In some embodiments, the subject had been administered an antibiotic prior to administration of the composition. In some embodiments, the antibiotic was administered less than a month, less than 30, 28, 21, 14, 13, 12, 11, 10, 9, 8, 7, 6, 5, 4, 3, 2 or 1 days prior to administration of the composition.

In certain aspects, provided herein is a composition for inducing the production of Rorγ⁺Helios⁻ LP Tregs in a subject comprising a bacteria or a combination of bacteria that induce the production of Rorγ⁺Helios⁻ LP Tregs. In some embodiments, the composition further comprises a pharmaceutically acceptable carrier. In some embodiments, the composition is a food product supplemented with the bacteria. In some embodiments, the food product is or comprises a dairy product (e.g., yogurt, frozen yogurt, ice cream, milk or cheese). In some embodiments, the food product is a non-dairy food product. In some embodiments, the food product is a beverage. In some embodiments, the composition is formulated for oral administration. In some embodiments, the composition is formulated for rectal administration.

In certain aspects, provided herein is a method of making a composition for inducing the production of Rorγ⁺Helios⁻ LP Tregs in a subject comprising combining a bacteria that induces the production of Rorγ⁺Helios⁻ LP Tregs with a pharmaceutically acceptable carrier. In some embodiments, the composition is formulated for oral administration. In some embodiments, the composition is formulated for rectal administration.

In certain aspects, provided herein is a method of making a composition for inducing the production of Rorγ⁺Helios⁻ LP Tregs in a subject comprising combining a bacteria that induces the production of Rorγ⁺Helios⁻ LP Tregs with a food product. In some embodiments, the food product is or comprises a dairy product (e.g., yogurt, frozen yogurt, ice cream, milk or cheese). In some embodiments, the food product is a non-dairy food product. In some embodiments, the food product is a beverage.

In some embodiments of the compositions and methods described herein, the bacteria that induces the production of Rorγ⁺Helios⁻ LP Tregs is selected from a species of bacteria listed in FIG. 5. In some embodiments, the bacteria that induces the production of Rorγ⁺Helios⁻ LP Tregs does not belong to the Clostridia class. In some embodiments, the bacteria that induces the production of Rorγ⁺Helios⁻ LP Tregs is selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve. In some embodiments, the bacteria is selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum and Parabacteroides johnsonii. In some embodiments, the bacteria is selected from the group consisting of Bacteroides ovatus, Campylobacter jejuni, Staphylococcus saprophyticus, Enterococcus faecalis, Bacteroides thetaiotaomicron and Bacteroides vulgatus. In some embodiments, the bacteria is selected from the group consisting of Bacteroides ovatus, Campylobacter jejuni, Staphylococcus saprophyticus and Enterococcus faecalis. In some embodiments, the bacteria is Bacteroides ovatus. In some embodiments, the bacteria is Campylobacter jejuni. In some embodiments, the bacteria is Staphylococcus saprophyticus. In some embodiments, the bacteria is Enterococcus faecalis. In some embodiments, the bacteria are live, replication competent bacteria.

In some embodiments of the compositions and methods described herein, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum and Parabacteroides johnsonii. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from the group consisting of Bacteroides ovatus, Campylobacter jejuni, Staphylococcus saprophyticus, Enterococcus faecalis, Bacteroides thetaiotaomicron and Bacteroides vulgatus. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from the group consisting of Bacteroides ovatus, Campylobacter jejuni, Staphylococcus saprophyticus and Enterococcus faecalis. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria is Bacteroides ovatus. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition is Campylobacter jejuni. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition is Staphylococcus saprophyticus. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition is Enterococcus faecalis.

In some embodiments of the compositions and methods described herein, the bacteria that induces the production of Rorγ⁺Helios⁻ LP Tregs is selected from a strain of bacteria listed in FIG. 5. In some embodiments, the bacteria that induces the production of Rorγ⁺Helios⁻ LP Tregs does not belong to the Clostridia class. In some embodiments, the bacteria that induces the production of Rorγ⁺Helios⁻ LP Tregs is selected from the group consisting of Bacteroides ovatus_CL02T12C04, Campylobacter jejuni_AS-84-79, Staphylococcus saprophyticus DLK1, Enterococcus faecalis_HH22, Bacteroides thetaiotaomicron_ATCC29741, Bacteroides vulgatus_ATCC8482, Bacteroides uniformis ATCC8492, Enterococcus faecalis TX0104, Lactobacillus casei AO47, Bacteroides fragilis_CL03T00C08, Acinetobacter lwoffii_F78, Fusobacterium nucleatum F0419, Enterococcus faecalis OG1RF, Bacteroides thetaiotaomicron_ATCC29148, Parabacteroides johnsonii_CL02T12C29, Bacteroides oleiciplenus_DSM22535, Lactobacillus rhamnosus_LMS2-1, Bacteroides massiliensis DSM17679, Parabacteroides merdae_CL03T12C32, Fusobacterium mortiferum_AO16, Bifidobacterium breve_SK134, Bacteroides finegoldii_DSM17565 and Bacteroides fragilis 3_1_12. In some embodiments, the bacteria is selected from the group consisting of Bacteroides ovatus_CL02T12C04, Campylobacter jejuni_AS-84-79, Staphylococcus saprophyticus_DLK1, Enterococcus faecalis_HH22, Bacteroides thetaiotaomicron_ATCC29741, Bacteroides vulgatus_ATCC8482, Bacteroides uniformis ATCC8492, Enterococcus faecalis TX0104, Lactobacillus casei AO47, Bacteroides fragilis_CL03T00C08, Acinetobacter lwoffii_F78, Fusobacterium nucleatum_F0419, Enterococcus faecalis_OG1RF, Bacteroides thetaiotaomicron_ATCC29148 and Parabacteroides johnsonii_CL02T12C29. In some embodiments, the bacteria is selected from the group consisting of Bacteroides ovatus_CL02T12C04, Campylobacter jejuni_AS-84-79, Staphylococcus saprophyticus_DLK1, Enterococcus faecalis_HH22, Bacteroides thetaiotaomicron_ATCC29741 and Bacteroides vulgatus_ATCC8482. In some embodiments, the bacteria is selected from the group consisting of Bacteroides ovatus_CL02T12C04, Campylobacter jejuni_AS-84-79, Staphylococcus saprophyticus_DLK1 and Enterococcus faecalis_HH22. In some embodiments, the bacteria is Bacteroides ovatus_CL02T12C04. In some embodiments, the bacteria is Campylobacter jejuni_AS-84-79. In some embodiments, the bacteria is Staphylococcus saprophyticus_DLK1. In some embodiments, the bacteria is Enterococcus faecalis_HH22. In some embodiments, the bacteria are live, replication competent bacteria.

In some embodiments of the compositions and methods described herein, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from the group consisting of Bacteroides ovatus_CL02T12C04, Campylobacter jejuni_AS-84-79, Staphylococcus saprophyticus_DLK1, Enterococcus faecalis_HH22, Bacteroides thetaiotaomicron_ATCC29741, Bacteroides vulgatus_ATCC8482, Bacteroides uniformis_ATCC8492, Enterococcus faecalis_TX0104, Lactobacillus casei_AO47, Bacteroides fragilis_CL03T00C08, Acinetobacter lwoffii_F78, Fusobacterium nucleatum F0419, Enterococcus faecalis OG1RF, Bacteroides thetaiotaomicron_ATCC29148, Parabacteroides johnsonii_CL02T12C29, Bacteroides oleiciplenus_DSM22535, Lactobacillus rhamnosus_LMS2-1, Bacteroides massiliensis DSM17679, Parabacteroides merdae CL03T12C32, Fusobacterium mortiferum_AO16, Bifidobacterium breve_SK134, Bacteroides finegoldii_DSM17565 and Bacteroides fragilis 3_1_12. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from the group consisting of Bacteroides ovatus_CL02T12C04, Campylobacter jejuni_AS-84-79, Staphylococcus saprophyticus_DLK1, Enterococcus faecalis_HH22, Bacteroides thetaiotaomicron_ATCC29741, Bacteroides vulgatus_ATCC8482, Bacteroides uniformis_ATCC8492, Enterococcus faecalis_TX0104, Lactobacillus casei_AO47, Bacteroides fragilis_CL03T00C08, Acinetobacter lwoffii_F78, Fusobacterium nucleatum F0419, Enterococcus faecalis OG1RF, Bacteroides thetaiotaomicron_ATCC29148 and Parabacteroides johnsonii_CL02T12C29. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from the group consisting of Bacteroides ovatus_CL02T12C04, Campylobacter jejuni_AS-84-79, Staphylococcus saprophyticus_DLK1, Enterococcus faecalis_HH22, Bacteroides thetaiotaomicron_ATCC29741 and Bacteroides vulgatus_ATCC8482. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from the group consisting of Bacteroides ovatus_CL02T12C04, Campylobacter jejuni_AS-84-79, Staphylococcus saprophyticus_DLK1 and Enterococcus faecalis_HH22. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition is Bacteroides ovatus CL02T12C04. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition is Campylobacter jejuni_AS-84-79. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition is Staphylococcus saprophyticus_DLK1. In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition is Enterococcus faecalis HH22.

In certain aspects, provided herein are methods and compositions for inducing the production or activity of regulatory T cells in a subject. In some embodiments, the methods comprise administering to the subject an agent that activates Rorγ (i.e., a Rorγ agonist). In some embodiments, the regulatory T cells are lamina propria regulatory T cells.

In certain aspects, provided herein are methods and compositions for treating or preventing a disease associated with a pathological immune response. In some embodiments, the methods comprise administering to the subject an agent that activates Rorγ (i.e., a Rorγ agonist). In some embodiments, the agent is administered in an amount sufficient to induce the production or activity of regulatory T cells (e.g., lamina propria regulatory T cells) in the subject. In some embodiments, the disease associated with a pathological immune response is an inflammatory bowel disease. In some embodiments, the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease. In some embodiments, the inflammatory bowel disease is Crohn's disease or ulcerative colitis.

In some embodiments of the methods described herein, a Rorγ agonist is administered to a subject (e.g., an effective dose of a Rorγ agonist). In some embodiments, the Rorγ agonist is a small molecule. In some embodiments, the Rorγ agonist is a sterol. In some embodiments, the sterol Rorγ agonist is selected from the group consisting of cholesterol sulfate, 25-OHC, 25-OHC sulfate, desmosterol, desmosterol sulfate, 5α,6α-epoxycholestanol sulfate, 7α,27-diOHC, 5α,6α-epoxycholestanol, 24S,25-epoxycholesterol, 7α-OHC, 20α-OHC, 22R-OHC, 24S-OHC, 27-OHC, 7β,27-OHC, 7-keto,27-OHC, 7α,27-OHC, 4AC4MΔ°, Δ7-daf, zymosterol, 4C, 7DHC, zymosterone, 4C22OH, 4,7-cholesten, 25OH, OR-12872 and OR-942

### BRIEF DESCRIPTION OF THE DRAWINGS

FIG. 1 shows Rorγ, encoded by Rorc, is preferentially expressed in colonic Tregs. (A) Heatmap of gene clusters that are enriched in tissue Tregs and colonic Tregs. Top: Genes that are overrepresented or underrepresented in tissue Tregs vs. splenic Tregs (at a Fold Change>2) were clustered using hierarchical clustering (Pearson correlation). Bottom: Heatmap of colon preferential gene clusters that are biased in colonic Tregs (at a Fold Change>1.5) compared with other tissue Tregs. (B) Heatmap of transcription factors that are overrepresented in colonic Tregs compared with Tregs from other lymphoid and non-lymphoid tissues. Rorc was the most differential based on hierarchical clustering analysis. (C) Representative dot plots (left) and respective frequencies (right) of Rorγ⁺Helios⁻ Tregs within the Foxp3⁺CD4⁺TCRβ⁺ population. (n=26) ***P<0.0001, paired t test. (D) Representative dot plots of Rorγ vs Helios, Nrp1 or Il33R expression within the colonic Foxp3⁺CD4⁺TCRβ⁺ population. Quantification of these plots is shown in FIG. S2 (n≥5). (E) depicts representative dot plots (top) and frequency (bottom) of Rorγ⁺Helios⁻ Tregs within the Foxp3⁺CD4⁺TCRβ⁺ population across different tissues—colon, small intestinal lamina propria (SI), Peyer's patches (PP), mesenteric lymph nodes (MLN), peripheral lymph nodes (LN), spleen (Spl), injured muscle, thymus and MC138 tumor. (F) Lymphocytes were treated with PMA and ionomycin and stained for Il17-A, Il17-F and FoxP3. Representative dot plots of Il17-A and Foxp3 expression (top), and frequencies of Il17-A or Il17-F producing cells (bottom) within Foxp3⁺ Tregs or Foxp3⁻ Tconvs from the colon (n=16), SI (n=4) and spleen (n=10) are shown.

FIG. 2 shows a comparison of gene expression profiles of Tregs from colon and spleen uncovered 933 differential transcripts. Colonic and splenic Tregs were sorted from Foxp3igfp mice. Fold change difference and corresponding p values between gene expression of colonic and splenic Tregs are shown in volcano plot. Colonic signature is highlighted at a FoldChange>2 and p value<0.05.

FIG. 3 shows Rorγ⁺ Tregs express low levels of Nrp1, Helios and IL33R. (A) Colonic lymphocytes were stained with Foxp3, Helios, Nrp1 and Rorγ. Frequencies of Helios⁺ or Nrp⁺ cells are reported within the Rorγ⁺ Tregs or Rorγ⁻ Tregs. (B) Representative dot plots of IL33R and Foxp3 expression within the CD4⁺TCRβ⁺ population and IL33R and Helios expression within the Foxp3⁺CD4⁺TCRβ⁺ population.

FIG. 4 shows Rorγ⁺Helios⁻ Tregs are induced by gut microbacteria. (A) Graphical representation of colonic Rorγ⁺Helios⁻ Tregs within the Foxp3⁺CD4⁺TCRβ⁺ population from adult SPF (n=14) and germ free mice (n=26). ***P<0.0001, unpaired t test. (B) Induction of Rorγ in colonic Tregs during normal post-natal development in SPF mice. Left: Representative dot plots of Rorγ⁺Helios⁻ and Rorγ⁻Helios⁻ Tregs within the Foxp3⁺CD4⁺TCRβ⁺ population before (d14) during (d19) and after (d24) Rorγ induction. Right top: Frequency of Foxp3⁺ Tregs within CD4⁺TCRβ⁺ cells. Right bottom: Frequency of Rorγ⁺Helios⁻ Tregs (red) and Rorγ⁻Helios⁻ Tregs (black) within the Foxp3⁺CD4⁺TCRβ⁺ population. (C) Mice were treated with single (Neomycin, Vancomycin, Ampicillin, Metronidazole) or full cocktail (VMNA) of antibiotics for 4 weeks. Frequency of colonic Rorγ⁺Helios⁻ Tregs within the Foxp3⁺CD4⁺TCRβ⁺ population is shown. ***P<0.0001, unpaired t test. (D) Germ free mice were colonized with single bacterial species (mono-colonization) for 2 weeks. Representative dot plots (top) and frequency of Rorγ⁺Helios⁻ Tregs (bottom) within the colonic Foxp3⁺CD4⁺TCRβ⁺ population following mono-colonization are shown. Coloring corresponds to different phyla. (E) Mice were mono-colonized with a focused sequenced set of Bacteroides. The frequency of Rorγ⁺Helios⁻ Tregs within the colonic Foxp3⁺CD4⁺TCRβ⁺ population is shown (E). (F) Colonic Rorγ⁺Helios⁻ Tregs and Rorγ⁻Helios⁻ Tregs were compared at different points of colonization in mice colonized with Bacteroides thetaiotaomicron ATCC29741. Representative dot plots (top) and respective frequencies within the Foxp3⁺CD4⁺TCRβ⁺ population (bottom) are shown.

FIG. 5 is a table showing the induction of colonic Rorγ⁺Helios⁻ Tregs by the indicated bacterial species. FDR refers to false discovery rate.

FIG. 6 shows induction of Rorγ⁻Helios⁻ Tregs is not accompanied by inflammation and is independent of bacterial load. (A) Haematoxylin and eosin staining of the distal colon from mono-colonized mice and the corresponding frequency of colonic Rorγ⁺Helios⁻ Tregs. (B) Correlation between bacterial load (measured as CFU) and frequency of colonic Rorγ⁺Helios⁻ Tregs in mono-colonized mice. Correlation coefficient=0.046.

FIG. 7 shows characterization of Treg populations in Foxp3-Cre. Rorc^{fl/fl} mice. Colonic and splenic lymphocytes were isolated from WT or Foxp3-Cre. Rorc^{fl/fl} mice, which lack Rorγ expression specifically in Tregs. Representative dot plots (left) and frequencies of Foxp3⁺ Tregs (middle) within the CD4⁺TCRP⁺ population and Helios⁻ Tregs (right) within the Foxp3⁺CD4⁺TCRβ⁺ population are shown. Foxp3, **P=0.004; Helios Tregs, *P=0.03, unpaired t test.

FIG. 8 shows Rorγ⁺ Tregs control colitis. (A) Colonic and splenic lymphocytes from WT (n=7) mice and Foxp3-Cre.Rorc^{fl/fl} (n=8) littermates were treated with PMA and ionomycin. Frequencies of Il17-A (left) and IFNγ (right) producing cells within the CD4⁺TCRβ⁺ population are shown. Il17-A, ***P=0.0002; IFNγ, **P=0.004, unpaired t test. (B) TNBS-colitis score of WT (n=9) and Foxp3-Cre.Rorc^{fl/fl} (n=8) littermates challenged with TNSB is shown. Colitis score is calculated based on weight loss, histologic score and other physical parameters (detailed in FIG. S5). **P=0.001, paired t test. (C) Haematoxylin and eosin staining of the distal colon. (D) Correlation of TNBS-colitis score (x-axis) with Rorγ⁺Helios⁻ Treg frequency (y-axis) in mice monocolonized for 2 weeks with bacteria that elicit high, medium or low Rorγ⁺Helios⁻ Treg phenotypes prior to the induction of TNBS-colitis. Correlation coefficient=0.82, ***P>0.0001.

FIG. 9 shows Rorγ Tregs protect from colitis. TNBS-colitis was induced in WT (n=9) and Foxp3-Cre.Rorc^{fl/fl} (n=8) littermates. Percent weight loss (left) and colon thickness (right) are shown. Data is representative of three independent experiments.

FIG. 10 shows Rorγ contributes to colonic Treg homeostasis and determines a part of the colonic Treg signature. (A) Colonic Rorγ⁺ or Rorγ⁻ Tregs were sorted from an intercross of Foxp3^{thy1.1} and Rorc^{gfp} reporter male mice. Mean gene expression values are shown for Rorγ⁺ or Rorγ⁻ Tregs and colonic Treg signature, shown in FIG. S1, is highlighted in red (induced) or blue (repressed) (n=3). (B) Some of the genes (Havcr2, Cxcr3), overrepresented in Rorγ⁺ Tregs, were validated at protein level. Representative dot plots of Cxcr3 and Tim3 encoded by Havcr2 are shown. (C) Fold change differences in gene expression between CD4 T cells and Treg cells that are enriched for or lack Rorγ expression are shown. CD4 T cells were sorted from SI of germ free mice or mice monocolonized with SFB. Gene changes that are specific to Rorγ⁺ Tregs (red), shared by both Rorγ⁺ Tregs and TH17 cells (green) and specific to TH17 cells (blue) are highlighted. (n=3).

### DETAILED DESCRIPTION

### General

In certain aspects, provided herein are methods and compositions related to the use of bacteria that induce the production of Rorγ⁺Helios⁻ lamina propria regulatory T cells (LP Tregs) for the production of such LP Tregs in a subject and/or the treatment or prevention an inflammatory bowel disease in a subject.

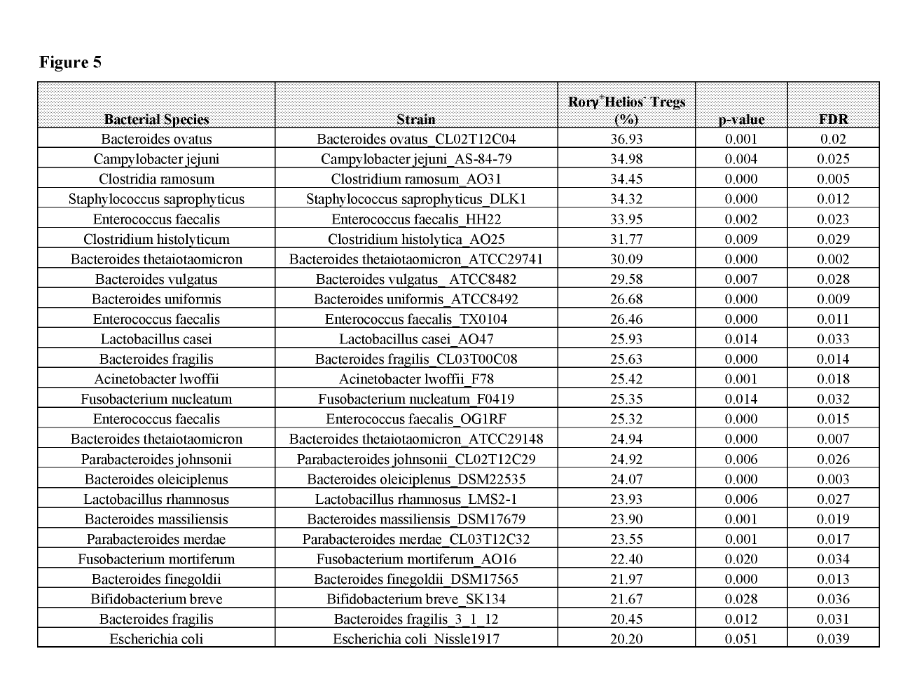

As described herein, the present inventors discovered that Rorγ is expressed in a FoxP3⁺Helios⁻ population of LP Tregs. Indeed, Rorγ⁺ Tregs are a major subset of those Tregs elicited in response to antigens of commensal microbes in the gut. The present inventors have further identified a number of human commensal bacterial species and strains that are capable of inducing production of Rorγ⁺Helios⁻ LP Tregs when administered to a subject. Examples of such bacteria are provided in FIG. 5 and include Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve. Compositions comprising such bacteria are therefore useful for the induction of Rorγ⁺Helios⁻ LP Tregs in a subject and/or for the treatment or prevention of diseases associated with a pathological immune response, such as an inflammatory bowel disease.

In certain aspects, provided herein are methods and compositions for the induction of lamina propria regulatory T cells (LP Tregs) and/or for the treatment of a disease associated with a pathological immune response (e.g., an inflammatory bowel disease) through the induction of Rorγ expression or activity. Thus, in certain aspects, provided herein are compositions and methods for the treatment and/or prevention of inflammatory bowel disease in a subject comprising administering to the subject an agent that activates Rorγ (i.e., an Rorγ agonist).

Rorγ is a transcription factor encoded by the Rorc gene that has previously been reported to be antagonistic to FoxP3 (Korn et al., Annu. Rev. Immunol. 27:485 (2009)). As FoxP3 is a key regulator of Treg development, inhibition of Rorγ has been proposed as a mechanism for the treatment of autoimmune diseases (e.g., U.S. Pat. No. 8,912,219, U.S. Pat. Pub. 2014/0163110, each of which is hereby incorporated by reference). Mice lacking FoxP3⁺ Treg expression of Rorγ have increased disease severity in a colitis model. This role for Rorγ contrasts strongly with the previously accepted dichotomy between FoxP3 and Rorγ, a notion that stems mainly from their antagonism in the outcome of TGFβ supplemented in vitro cultures (Zou et al., Nature 453:236 (2008)).

As described herein, in LP Tregs, Rorγ controls a specific transcriptional signature, overlapping but mainly distinct from its transcriptional signature in conventional T cells. Notably missing are most transcripts encoding the pro-inflammatory IL-17 cytokine family, but the shared aspects of the transcriptional signature include Il23r. Notably, human IL23R genetic variants are strongly associated with inflammatory bowel disease (Abraham and Cho, Annu. Rev. Med. 60:97 (2009)). Rorγ⁺ Tregs do not respond to the alarmin IL-33, and are phenotypically distinct from Il33R⁺Helios⁺ cells that congregate or expand in response to IL-33 in response to tissue damage. Mutually exclusive expression of IL-33 receptor and Rorγ in LP Tregs indicates that these molecules distinguish responses to commensal versus aggressive microbes.

### Definitions

For convenience, certain terms employed in the specification, examples, and appended claims are collected here.

The articles “a” and “an” are used herein to refer to one or to more than one (i.e., to at least one) of the grammatical object of the article.

As used herein, the term “administering” means providing an agent or composition to a subject, and includes, but is not limited to, administering by a medical professional and self-administering.

The term “agent” is used herein to denote a chemical compound, a small molecule, a mixture of chemical compounds and/or a biological macromolecule (such as a nucleic acid, an antibody, an antibody fragment, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

As used herein, an “effective amount” is an amount effective in treating or preventing a disease associated with a pathological immune response, including, for example, inflammatory bowel disease.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays described herein.

As used herein, the term “subject” means a human or non-human animal selected for treatment or therapy. In certain embodiments, of the methods and compositions described herein the subject is a human subject.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

Bacteria that Induce LP Tregs

In certain aspects, provided herein are compositions and methods related the use of bacteria that induce the production of Rorγ⁺Helios⁻ LP Tregs. Examples of such bacteria are provided in FIG. 5. In some embodiments, the bacteria does not belong to the Clostridia class.

In some embodiments, the species of the bacteria that induces the production of Rorγ⁺Helios⁻ LP Tregs is Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the strain of the bacteria that induces the production of Rorγ⁺Helios⁻ LP Tregs is selected from the group consisting of Bacteroides ovatus_CL02T12C04, Campylobacter jejuni_AS-84-79, Staphylococcus saprophyticus_DLK1, Enterococcus faecalis_HH22, Bacteroides thetaiotaomicron_ATCC29741, Bacteroides vulgatus_ATCC8482, Bacteroides uniformis ATCC8492, Enterococcus faecalis TX0104, Lactobacillus casei AO47, Bacteroides fragilis_CL03T00C08, Acinetobacter lwoffii_F78, Fusobacterium nucleatum_F0419, Enterococcus faecalis_OG1RF, Bacteroides thetaiotaomicron_ATCC29148, Parabacteroides johnsonii_CL02T12C29, Bacteroides oleiciplenus_DSM22535, Lactobacillus rhamnosus_LMS2-1, Bacteroides massiliensis DSM17679, Parabacteroides merdae CL03T12C32, Fusobacterium mortiferum_AO16, Bifidobacterium breve_SK134, Bacteroides finegoldii_DSM17565 and/or Bacteroides fragilis 3_1_12.

In some embodiments, the bacteria that induce the production of Rorγ⁺Helios⁻ LP Tregs do not express a heterologous gene (i.e., are not recombinant). In some embodiments, the bacteria that induce the production of Rorγ⁺Helios⁻ LP Tregs are recombinant. In some embodiments, the bacteria that induce the production of Rorγ⁺Helios⁻ LP Tregs are attenuated.

In some embodiments, the bacteria that induce the production of Rorγ⁺Helios⁻ LP Tregs has a genomic sequence at least 90%, 91%, 92%, 93%, 94%, 95%, 96%, 97%, 98%, 99% or 100% homology the genomic sequence of a bacteria strain described herein (e.g., Bacteroides ovatus_CL02T12C04, Campylobacter jejuni_AS-84-79, Staphylococcus saprophyticus_DLK1, Enterococcus faecalis_HH22, Bacteroides thetaiotaomicron_ATCC29741, Bacteroides vulgatus_ATCC8482, Bacteroides uniformis_ATCC8492, Enterococcus faecalis_TX0104, Lactobacillus casei_AO47, Bacteroides fragilis_CL03T00C08, Acinetobacter lwoffii_F78, Fusobacterium nucleatum F0419, Enterococcus faecalis OG1RF, Bacteroides thetaiotaomicron_ATCC29148, Parabacteroides johnsonii_CL02T12C29, Bacteroides oleiciplenus_DSM22535, Lactobacillus rhamnosus_LMS2-1, Bacteroides massiliensis DSM17679, Parabacteroides merdae CL03T12C32, Fusobacterium mortiferum_AO16, Bifidobacterium breve_SK134, Bacteroides finegoldii_DSM17565 and/or Bacteroides fragilis_3_1_12).

The bacteria described herein can be grown in culture using methods known in the art. For example, Bacteroides Clostridia, Bifidobacteria, Lactobacilli, Enterococci, Fusobacteria, Propionibacteria and Peptostreptococcus can be grown in supplemented Yeast extract-peptone-glycerol (YPG) medium, Blood Brucella Agar or Blood TSA Agar plates. Acinetobacter can be grown in SB medium and LB Agar plates. Lachnospiraceae, Veillonella, Coprobacillus can be grown in chopped meat broth. Anaerobic bacteria can be cultured under strictly anaerobic conditions (80% N₂, 10% H₂, 10% CO₂) at 37° C. in an anaerobic chamber. Staphylococcus can be grown aerobically, at 37° C. in L-broth and on LB Agar plates.

In some embodiments, combinations of species or strains of bacteria induce the production of Rorγ⁺Helios⁻ LP Tregs (e.g., the strains and species of bacteria listed in FIG. 5) are used in the methods and/or compositions provided herein. In certain embodiments, a combination of at least 2, 3, 4, 5, 6, 7, 8, 9, 10, 11, 12, 13, 14, 15, 16, 17, 18 or 19 of species or strains of bacteria induce the production of Rorγ⁺Helios⁻ LP Tregs are used in the methods and/or compositions described herein.

In some embodiments, the combination of bacteria used in the compositions and/or methods described herein includes Bacteroides ovatus and one or more bacterial strains selected from the group consisting of Campylobacter jejuni, Clostridia ramosum, Staphylococcus saprophyticus, Enterococcus faecalis, Clostridium histolyticum, Bacteroides thetaiotaomicron, Bacteroides vulgatus, Bacteroides uniformis, Lactobacillus casei, Bacteroides fagilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni and one or more bacterial strains selected from the group consisting of Clostridia ramosum, Staphylococcus saprophyticus, Enterococcus faecalis, Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum and one or more bacterial strains selected from the group consisting of Staphylococcus saprophyticus, Enterococcus faecalis, Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus and one or more bacterial strains selected from the group consisting of Enterococcus faecalis, Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis and one or more bacterial strains selected from the group consisting of Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum and one or more bacterial strains selected from the group consisting of Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron and one or more bacterial strains selected from the group consisting of Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides vulgatus and one or more bacterial strains selected from the group consisting of Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides uniformis and one or more bacterial strains selected from the group consisting of Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Lactobacillus casei and one or more bacterial strains selected from the group consisting of Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Parabacteroides merdae and one or more bacterial strains selected from the group consisting of Fusobacterium mortiferum, Bacteroides finegoldii and/or Bifidobacterium breve.

In some embodiments, the combination of bacteria includes Fusobacterium mortiferum and one or more bacterial strains selected from the group consisting of Bacteroides finegoldii and/or Bifidobacterium breve.

In some embodiments, the combination of bacteria includes Bacteroides finegoldii and Bifidobacterium breve.

In some embodiments, the combination of bacteria includes Bacteroides ovatus, Campylobacter jejuni and one or more bacterial strains selected from the group consisting of Clostridia ramosum, Staphylococcus saprophyticus, Enterococcus faecalis, Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Clostridia ramosum and one or more bacterial strains selected from the group consisting of Staphylococcus saprophyticus, Enterococcus faecalis, Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Staphylococcus saprophyticus and one or more bacterial strains selected from the group consisting of Enterococcus faecalis, Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Enterococcus faecalis and one or more bacterial strains selected from the group consisting of Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum and one or more bacterial strains selected from the group consisting of Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Bacteroides thetaiotaomicron and one or more bacterial strains selected from the group consisting of Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Bacteroides vulgatus and one or more bacterial strains selected from the group consisting of Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Bacteroides uniformis and one or more bacterial strains selected from the group consisting of Lactobacillus casei, Bacteroides fragilis, Acinetobacter woffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Lactobacillus casei and one or more bacterial strains selected from the group consisting of Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Bacteroides ovatus, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Bacteroides ovatus, Parabacteroides merdae and one or more bacterial strains selected from the group consisting of Fusobacterium mortiferum, Bacteroides finegoldii and/or Bifidobacterium breve.

In some embodiments, the combination of bacteria includes Bacteroides ovatus, Fusobacterium mortiferum and one or more bacterial strains selected from the group consisting of Bacteroides finegoldii and/or Bifidobacterium breve.

In some embodiments, the combination of bacteria includes Bacteroides ovatus, Bacteroides finegoldii and Bifidobacterium breve.

In some embodiments, the combination of bacteria includes Campylobacter jejuni, Clostridia ramosum and one or more bacterial strains selected from the group consisting of Staphylococcus saprophyticus, Enterococcus faecalis, Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Staphylococcus saprophyticus and one or more bacterial strains selected from the group consisting of Enterococcus faecalis, Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Enterococcus faecalis and one or more bacterial strains selected from the group consisting of Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Clostridium histolyticum and one or more bacterial strains selected from the group consisting of Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Bacteroides thetaiotaomicron and one or more bacterial strains selected from the group consisting of Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Bacteroides vulgatus and one or more bacterial strains selected from the group consisting of Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Bacteroides uniformis and one or more bacterial strains selected from the group consisting of Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Lactobacillus casei and one or more bacterial strains selected from the group consisting of Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Campylobacter jejuni, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Campylobacter jejuni, Parabacteroides merdae and one or more bacterial strains selected from the group consisting of Fusobacterium mortiferum, Bacteroides finegoldii and/or Bifidobacterium breve.

In some embodiments, the combination of bacteria includes Campylobacter jejuni, Fusobacterium mortiferum and one or more bacterial strains selected from the group consisting of Bacteroides finegoldii and/or Bifidobacterium breve.

In some embodiments, the combination of bacteria includes Campylobacter jejuni, Bacteroides finegoldii and Bifidobacterium breve.

In some embodiments, the combination of bacteria includes Clostridia ramosum, Staphylococcus saprophyticus and one or more bacterial strains selected from the group consisting of Enterococcus faecalis, Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Enterococcus faecalis and one or more bacterial strains selected from the group consisting of Clostridium histolyticum, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Clostridium histolyticum and one or more bacterial strains selected from the group consisting of Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Bacteroides thetaiotaomicron and one or more bacterial strains selected from the group consisting of Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Bacteroides vulgatus and one or more bacterial strains selected from the group consisting of Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Bacteroides uniformis and one or more bacterial strains selected from the group consisting of Lactobacillus casei, Bacteroides fragilis, Acinetobacter woffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Lactobacillus casei and one or more bacterial strains selected from the group consisting of Bacteroides fagilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Bacteroides fagilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Clostridia ramosum, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Clostridia ramosum, Parabacteroides merdae and one or more bacterial strains selected from the group consisting of Fusobacterium mortiferum, Bacteroides finegoldii and/or Bifidobacterium breve.

In some embodiments, the combination of bacteria includes Clostridia ramosum, Fusobacterium mortiferum and one or more bacterial strains selected from the group consisting of Bacteroides finegoldii and/or Bifidobacterium breve.

In some embodiments, the combination of bacteria includes Clostridia ramosum, Bacteroides finegoldii and Bifidobacterium breve.

In some embodiments, the combination of bacteria includes Enterococcus faecalis and one or more bacterial strains selected from the group consisting of Clostridium histolyticum, Bacteroides thetaiotaomicron, Bacteroides vulgatus, Bacteroides uniformis, Lactobacillus casei, Bacteroides fagilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Clostridium histolyticum and one or more bacterial strains selected from the group consisting of Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Bacteroides thetaiotaomicron and one or more bacterial strains selected from the group consisting of Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Bacteroides vulgatus and one or more bacterial strains selected from the group consisting of Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Bacteroides uniformis and one or more bacterial strains selected from the group consisting of Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Lactobacillus casei and one or more bacterial strains selected from the group consisting of Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Staphylococcus saprophyticus, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Staphylococcus saprophyticus, Parabacteroides merdae and one or more bacterial strains selected from the group consisting of Fusobacterium mortiferum, Bacteroides finegoldii and/or Bifidobacterium breve.

In some embodiments, the combination of bacteria includes Staphylococcus saprophyticus, Fusobacterium mortiferum and one or more bacterial strains selected from the group consisting of Bacteroides finegoldii and/or Bifidobacterium breve.

In some embodiments, the combination of bacteria includes Staphylococcus saprophyticus, Bacteroides finegoldii and Bifidobacterium breve.

In some embodiments, the combination of bacteria includes Enterococcus faecalis, Clostridium histolyticum and one or more bacterial strains selected from the group consisting of Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Bacteroides thetaiotaomicron and one or more bacterial strains selected from the group consisting of Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Bacteroides vulgatus and one or more bacterial strains selected from the group consisting of Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Bacteroides uniformis and one or more bacterial strains selected from the group consisting of Lactobacillus casei, Bacteroides fragilis, Acinetobacter woffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Lactobacillus casei and one or more bacterial strains selected from the group consisting of Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Enterococcus faecalis, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Enterococcus faecalis, Parabacteroides merdae and one or more bacterial strains selected from the group consisting of Fusobacterium mortiferum, Bacteroides finegoldii and/or Bifidobacterium breve.

In some embodiments, the combination of bacteria includes Enterococcus faecalis, Fusobacterium mortiferum and one or more bacterial strains selected from the group consisting of Bacteroides finegoldii and/or Bifidobacterium breve.

In some embodiments, the combination of bacteria includes Enterococcus faecalis, Bacteroides finegoldii and Bifidobacterium breve.

In some embodiments, the combination of bacteria includes Clostridium histolyticum, Bacteroides thetaiotaomicron and one or more bacterial strains selected from the group consisting of Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Bacteroides vulgatus and one or more bacterial strains selected from the group consisting of Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Bacteroides uniformis and one or more bacterial strains selected from the group consisting of Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Lactobacillus casei and one or more bacterial strains selected from the group consisting of Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Clostridium histolyticum, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Clostridium histolyticum, Parabacteroides merdae and one or more bacterial strains selected from the group consisting of Fusobacterium mortiferum, Bacteroides finegoldii and/or Bifidobacterium breve.

In some embodiments, the combination of bacteria includes Clostridium histolyticum, Fusobacterium mortiferum and one or more bacterial strains selected from the group consisting of Bacteroides finegoldii and/or Bifidobacterium breve.

In some embodiments, the combination of bacteria includes Clostridium histolyticum, Bacteroides finegoldii and Bifidobacterium breve.

In some embodiments, the combination of bacteria includes Bacteroides thetaiotaomicron, Bacteroides vulgatus and one or more bacterial strains selected from the group consisting of Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron, Bacteroides uniformis and one or more bacterial strains selected from the group consisting of Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron, Lactobacillus casei and one or more bacterial strains selected from the group consisting of Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron, Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron, Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Bacteroides thetaiotaomicron, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Bacteroides thetaiotaomicron, Parabacteroides merdae and one or more bacterial strains selected from the group consisting of Fusobacterium mortiferum, Bacteroides finegoldii and/or Bifidobacterium breve.

In some embodiments, the combination of bacteria includes Bacteroides thetaiotaomicron, Fusobacterium mortiferum and one or more bacterial strains selected from the group consisting of Bacteroides finegoldii and/or Bifidobacterium breve.

In some embodiments, the combination of bacteria includes Bacteroides thetaiotaomicron, Bacteroides finegoldii and Bifidobacterium breve.

In some embodiments, the combination of bacteria includes Bacteroides vulgatus, Bacteroides uniformis and one or more bacterial strains selected from the group consisting of Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides vulgatus, Lactobacillus casei and one or more bacterial strains selected from the group consisting of Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides vulgatus, Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides vulgatus, Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides vulgatus, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides vulgatus, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides vulgatus, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides vulgatus, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Bacteroides vulgatus, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Bacteroides vulgatus, Parabacteroides merdae and one or more bacterial strains selected from the group consisting of Fusobacterium mortiferum, Bacteroides finegoldii and/or Bifidobacterium breve.

In some embodiments, the combination of bacteria includes Bacteroides vulgatus, Fusobacterium mortiferum and one or more bacterial strains selected from the group consisting of Bacteroides finegoldii and/or Bifidobacterium breve.

In some embodiments, the combination of bacteria includes Bacteroides vulgatus, Bacteroides finegoldii and Bifidobacterium breve.

In some embodiments, the combination of bacteria includes Bacteroides uniformis, Lactobacillus casei and one or more bacterial strains selected from the group consisting of Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides uniformis, Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides uniformis, Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides uniformis, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides uniformis, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides uniformis, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides uniformis, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Bacteroides uniformis, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Bacteroides uniformis, Parabacteroides merdae and one or more bacterial strains selected from the group consisting of Fusobacterium mortiferum, Bacteroides finegoldii and/or Bifidobacterium breve.

In some embodiments, the combination of bacteria includes Bacteroides uniformis, Fusobacterium mortiferum and one or more bacterial strains selected from the group consisting of Bacteroides finegoldii and/or Bifidobacterium breve.

In some embodiments, the combination of bacteria includes Bacteroides uniformis, Bacteroides finegoldii and Bifidobacterium breve.

In some embodiments, the combination of bacteria includes Lactobacillus casei, Bacteroides fragilis and one or more bacterial strains selected from the group consisting of Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Lactobacillus casei, Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Lactobacillus casei, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Lactobacillus casei, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Lactobacillus casei, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Lactobacillus casei, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Lactobacillus casei, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Lactobacillus casei, Parabacteroides merdae and one or more bacterial strains selected from the group consisting of Fusobacterium mortiferum, Bacteroides finegoldii and/or Bifidobacterium breve.

In some embodiments, the combination of bacteria includes Lactobacillus casei, Fusobacterium mortiferum and one or more bacterial strains selected from the group consisting of Bacteroides finegoldii and/or Bifidobacterium breve.

In some embodiments, the combination of bacteria includes Lactobacillus casei, Bacteroides finegoldii and Bifidobacterium breve.

In some embodiments, the combination of bacteria includes Bacteroides fagilis, Acinetobacter lwoffii and one or more bacterial strains selected from the group consisting of Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides fagilis, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides fagilis, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides fagilis, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Bacteroides fagilis, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Bacteroides fagilis, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Bacteroides fagilis, Parabacteroides merdae and one or more bacterial strains selected from the group consisting of Fusobacterium mortiferum, Bacteroides finegoldii and/or Bifidobacterium breve.

In some embodiments, the combination of bacteria includes Bacteroides fagilis, Fusobacterium mortiferum and one or more bacterial strains selected from the group consisting of Bacteroides finegoldii and/or Bifidobacterium breve.

In some embodiments, the combination of bacteria includes Bacteroides fagilis, Bacteroides finegoldii and Bifidobacterium breve.

In some embodiments, the combination of bacteria includes Acinetobacter lwoffii, Fusobacterium nucleatum and one or more bacterial strains selected from the group consisting of Parabacteroides johnsonii, Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Acinetobacter lwoffii, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Acinetobacter lwoffii, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Acinetobacter lwoffii, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Acinetobacter lwoffii, Parabacteroides merdae and one or more bacterial strains selected from the group consisting of Fusobacterium mortiferum, Bacteroides finegoldii and/or Bifidobacterium breve.

In some embodiments, the combination of bacteria includes Acinetobacter lwoffii, Fusobacterium mortiferum and one or more bacterial strains selected from the group consisting of Bacteroides finegoldii and/or Bifidobacterium breve.

In some embodiments, the combination of bacteria includes Acinetobacter lwoffii, Bacteroides finegoldii and Bifidobacterium breve.

In some embodiments, the combination of bacteria includes Fusobacterium nucleatum, Parabacteroides johnsonii and one or more bacterial strains selected from the group consisting of Bacteroides oleiciplenus, Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Fusobacterium nucleatum, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Fusobacterium nucleatum, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Fusobacterium nucleatum, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Fusobacterium nucleatum, Parabacteroides merdae and one or more bacterial strains selected from the group consisting of Fusobacterium mortiferum, Bacteroides finegoldii and/or Bifidobacterium breve.

In some embodiments, the combination of bacteria includes Fusobacterium nucleatum, Fusobacterium mortiferum and one or more bacterial strains selected from the group consisting of Bacteroides finegoldii and/or Bifidobacterium breve.

In some embodiments, the combination of bacteria includes Fusobacterium nucleatum, Bacteroides finegoldii and Bifidobacterium breve.

In some embodiments, the combination of bacteria includes Parabacteroides johnsonii, Bacteroides oleiciplenus and one or more bacterial strains selected from the group consisting of Lactobacillus rhamnosus, Bacteroides massiliensis, Parabacteroides merdae, Fusobacterium mortiferum, Bacteroides finegoldii and/or Bifidobacterium breve.

In some embodiments, the combination of bacteria includes Parabacteroides johnsonii, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Parabacteroides johnsonii, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Parabacteroides johnsonii, Parabacteroides merdae and one or more bacterial strains selected from the group consisting of Fusobacterium mortiferum, Bacteroides finegoldii and/or Bifidobacterium breve.

In some embodiments, the combination of bacteria includes Parabacteroides johnsonii, Fusobacterium mortiferum and one or more bacterial strains selected from the group consisting of Bacteroides finegoldii and/or Bifidobacterium breve.

In some embodiments, the combination of bacteria includes Parabacteroides johnsonii, Bacteroides finegoldii and Bifidobacterium breve.

In some embodiments, the combination of bacteria includes Bacteroides oleiciplenus, Lactobacillus rhamnosus and one or more bacterial strains selected from the group consisting of Bacteroides massiliensis, Parabacteroides merdae, Fusobacterium mortiferum, Bacteroides finegoldii and/or Bifidobacterium breve.

In some embodiments, the combination of bacteria includes Bacteroides oleiciplenus, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Bacteroides oleiciplenus, Parabacteroides merdae and one or more bacterial strains selected from the group consisting of Fusobacterium mortiferum, Bacteroides finegoldii and/or Bifidobacterium breve.

In some embodiments, the combination of bacteria includes Bacteroides oleiciplenus, Fusobacterium mortiferum and one or more bacterial strains selected from the group consisting of Bacteroides finegoldii and/or Bifidobacterium breve.

In some embodiments, the combination of bacteria includes Bacteroides oleiciplenus, Bacteroides finegoldii and Bifidobacterium breve.

In some embodiments, the combination of bacteria includes Lactobacillus rhamnosus, Bacteroides massiliensis and one or more bacterial strains selected from the group consisting of Parabacteroides merdae, Fusobacterium mortiferum, Bacteroides finegoldii and/or Bifidobacterium breve.

In some embodiments, the combination of bacteria includes Lactobacillus rhamnosus, Parabacteroides merdae and one or more bacterial strains selected from the group consisting of Fusobacterium mortiferum, Bacteroides finegoldii and/or Bifidobacterium breve.

In some embodiments, the combination of bacteria includes Lactobacillus rhamnosus, Fusobacterium mortiferum and one or more bacterial strains selected from the group consisting of Bacteroides finegoldii and/or Bifidobacterium breve.

In some embodiments, the combination of bacteria includes Lactobacillus rhamnosus, Bacteroides finegoldii and Bifidobacterium breve.

In some embodiments, the combination of bacteria includes Bacteroides massiliensis, Parabacteroides merdae and one or more bacterial strains selected from the group consisting of Fusobacterium mortiferum, Bacteroides finegoldii and/or Bifidobacterium breve.

In some embodiments, the combination of bacteria includes Bacteroides massiliensis, Fusobacterium mortiferum and one or more bacterial strains selected from the group consisting of Bacteroides finegoldii and/or Bifidobacterium breve.

In some embodiments, the combination of bacteria includes Bacteroides massiliensis, Bacteroides finegoldii and Bifidobacterium breve.

In some embodiments, the combination of bacteria includes Parabacteroides merdae, Fusobacterium mortiferum and one or more bacterial strains selected from the group consisting of Bacteroides finegoldii and/or Bifidobacterium breve.

In some embodiments, the combination of bacteria includes Parabacteroides merdae, Bacteroides finegoldii and Bifidobacterium breve.

In some embodiments, the combination of bacteria includes Fusobacterium mortiferum, Bacteroides finegoldii and Bifidobacterium breve.

### Bacterial Compositions

In certain embodiments, provided herein is a composition (e.g., a pharmaceutical composition, a dietary supplement or a food product) containing bacteria or combinations of bacteria that induce the production of Rorγ⁺Helios⁻ LP Tregs. In some embodiments, the composition further comprises a pharmaceutically acceptable carrier.

In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from among the bacterial species described herein. 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from among the bacterial strains described herein.

In some embodiments, the compositions described herein may include only one species of bacteria described herein or may include two or more species of the bacteria described herein. For example, 1, 2, 3, 4, 5, 6, 7, 8, 9, 10, 11, 12, 13, 14, 15, 16, 17, 18, 19 or 20 of the species described herein, in any combination, can be included in the compositions provided herein.

In some embodiments, the compositions described herein may include only one strain of the bacteria described herein or may include two or more strains of the bacteria described herein. For example, 1, 2, 3, 4, 5, 6, 7, 8, 9, 10, 11, 12, 13, 14, 15, 16, 17, 18, 19 or 20 of the strains described herein, in any combination, can be included in the compositions provided herein.

In some embodiments, the composition described herein may be a pharmaceutical composition, a dietary supplement, or a food product (e.g., a food or beverage). In some embodiments, the food product is an animal feed.

As described in detail below, the pharmaceutical compositions disclosed herein may be specially formulated for administration in solid or liquid form, including those adapted for oral or rectal administration.

In certain embodiments, the pharmaceutical composition for oral administration described herein comprises an additional component that enables efficient delivery of the bacteria to the colon, in order to more efficiently induce the production of LP Tregs in the colon. In some embodiments, pharmaceutical preparation that enables the delivery of the bacteria to the colon can be used. Examples of such formulations include pH sensitive compositions, such as buffered sachet formulations or enteric polymers that release their contents when the pH becomes alkaline after the enteric polymers pass through the stomach. When a pH sensitive composition is used for formulating the pharmaceutical preparation, the pH sensitive composition can be a polymer whose pH threshold of the decomposition of the composition is between about 6.8 and about 7.5.

Another embodiment of a pharmaceutical composition useful for delivery of the bacteria to the colon is one that ensures the delivery to the colon by delaying the release of the bacteria by approximately 3 to 5 hours, which corresponds to the small intestinal transit time. In some embodiments, the pharmaceutical composition for delayed release includes a hydrogel shell. The hydrogel is hydrated and swells upon contact with gastrointestinal fluid, with the result that the contents are effectively released (released predominantly in the colon). Delayed release dosage units include bacteria-containing compositions having a material which coats or selectively coats the bacteria. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Examples of composition enabling the delivery to the colon further include bioadhesive compositions which specifically adhere to the colonic mucosal membrane (for example, a polymer described in the specification of U.S. Pat. No. 6,368,586, hereby incorporated by reference) and compositions into which a protease inhibitor is incorporated for protecting particularly a biopharmaceutical preparation in the gastrointestinal tracts from decomposition due to an activity of a protease.

An example of a system enabling the delivery to the colon is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

Another example of the system enabling the delivery to the colon is a system of delivering a composition to the colon, the system being specifically decomposed by an enzyme (for example, a carbohydrate hydrolase or a carbohydrate reductase) present in the colon. Such a system is not particularly limited, and more specific examples thereof include systems which use food components such as non-starch polysaccharides, amylose, xanthan gum, and azopolymers.

In some embodiments, the compositions described herein further comprise an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments the composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Rorγ

In certain embodiments, provided herein are methods inducing LP Tregs and/or treating a disease or disorder associated with a pathological immune response, such as an inflammatory bowel disease, by inducing Rorγ. Activation of Rorγ can, for example, be via an increase in Rorγ protein activity or Rorγ protein amount. For example, agents that induce Rorγ include agents that increase Rorγ protein activity, agents that decrease Rorγ protein degradation, agents that increase Rorγ mRNA stability, and agents that increase transcription and/or translation of nucleic acids encoding Rorγ protein.

Rorγ is a DNA-binding transcription factor and is a member of the NR1 subfamily of nuclear hormone receptors. Rorγ is encoded by the Rorc gene. While Rorγ has previously been reported to be antagonistic to Treg inducing transcription factor FoxP3, as disclosed herein, Rorγ is expressed in a major population of LP Tregs. The amino acid sequence human Rorγ is available at NCBI accession number XP_006711547.2, which is incorporated by reference herein. The nucleic acid sequence of the human Rorγ isoform mRNA is available at NCBI accession numbers XM_006711484.2, which is incorporated by reference herein.

### Rorγ Agonists

In certain embodiments, provided herein are compositions and methods for inducing Tregs and/or for treating inflammatory bowel disease. These methods include administering an agent that activates Rorγ (i.e., a Rorγ agonist). Such agents include those disclosed below, those known in the art and those identified using the screening assays described herein. In some embodiments, any agent that activates Rorγ can be used to practice the methods disclosed herein. Rorγ agonists may be small molecules, proteins, peptides, nucleic acids, carbohydrates or antibodies. In some embodiments, the Rorγ agonist is a sterol. Exemplary Rorγ agonists are described in Hu et al., Nature Chemical Biology 11:141-147 (2015), Soroosh et al., Proc. Natl. Acad. Sci. USA 111:12163-12168 (2014) and Santori et al., Cell Metabolism 21:286-297 (2015) and U.S. Pat. No. 8,389,739, each of which is hereby incorporated by reference.

In some embodiments, the Rorγ agonist is a sterol selected from the group consisting of cholesterol sulfate, 25-OHC, 25-OHC sulfate, desmosterol, desmosterol sulfate, 5α,6α-epoxycholestanol sulfate, 7α,27-diOHC, 5α,6α-epoxycholestanol, 24S,25-epoxycholesterol, 7α-OHC, 20α-OHC, 22R-OHC, 24S-OHC, 27-OHC, 7β,27-OHC, 7-keto,27-OHC, 7α,27-OHC, 4AC4MΔ°, Δ7-daf, zymosterol, 4C, 7DHC, zymosterone, 4C22OH, 4,7-cholesten, 25OH, OR-12872, OR-942, or a prodrug, active derivative or pharmaceutically acceptable salt thereof.

In some embodiments, the Rorγ agonist is 25-OHC, a 25-OHC prodrug, an active derivative of 25-OHC or a pharmaceutically acceptable salt thereof. 25-OHC has the following chemical structure:

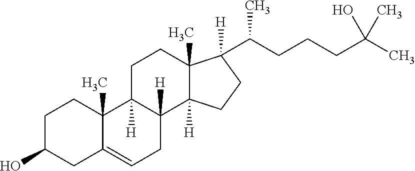

In some embodiments, the Rorγ agonist is hyodeoxycholic acid methyl ester (OR-942), a hyodeoxycholic acid methyl ester prodrug, an active derivative of hyodeoxycholic acid methyl ester or a pharmaceutically acceptable salt thereof. hyodeoxycholic acid methyl ester has the following chemical structure:

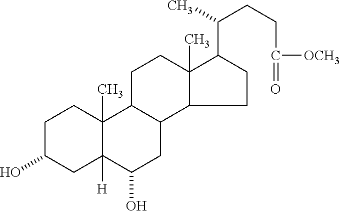 [SMILES: COC(=O)CC[C@@H](C)C1CCC2C3C[C@H](O)C4C[C@H](O)CCC4(C)C3CCC21C]

In some embodiments, the Rorγ agonist is OR-12872 (4R-[3R,6R-Bis-(tert-butyl-dimethyl-silanyloxy)-10R, 13R-dimethyl-5R-8S-9S-14 S-hexadecahydrocyclopenta[a]phenanthren-17R-yl]-pentanoic acid methyl ester), an OR-12872 prodrug, an active derivative of OR-12872 or a pharmaceutically acceptable salt thereof. Synthesis of OR-12872 is described in U.S. Pat. No. 8,389,739, which is hereby incorporated by reference in its entirety. OR-12872 has the following chemical structure:

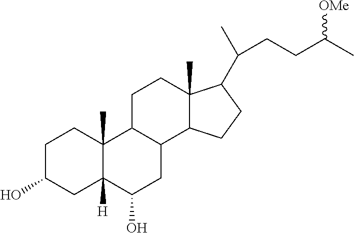 [SMILES: COC(C)CCC(C)C1CCC2C3C[C@H](O)[C@@H]4C[C@H](O)CC[C@]4(C)C3CC[C@]12C]

In some embodiments, the Rorγ agonist is desmosterol, a desmosterol prodrug, an active derivative of desmosterol or a pharmaceutically acceptable salt thereof. Desmosterol has the following chemical structure:

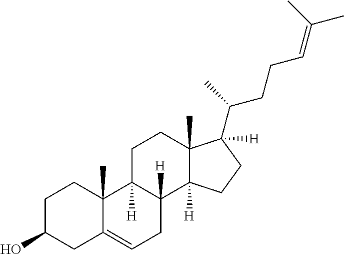

In some embodiments, the Rorγ agonist is 7β,27-OHC, a 7β,27-OHC prodrug, an active derivative of 7β,27-OHC or a pharmaceutically acceptable salt thereof. 7β,27-OHC has the following chemical structure:

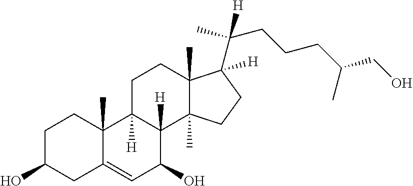

In some embodiments, the Rorγ agonist is 7keto,27-OHC, a 7keto,27-OHC prodrug, an active derivative of 7keto,27-OHC or a pharmaceutically acceptable salt thereof. 7keto,27-OHC has the following chemical structure:

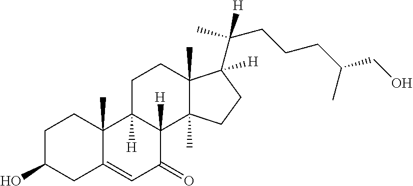

In some embodiments, the Rorγ agonist is 4α-carboxy,4β-methyl-zymosterol (4ACD8), a 4ACD8 prodrug, an active derivative of 4ACD8 or a pharmaceutically acceptable salt thereof. 4ACD8 has the following chemical structure:

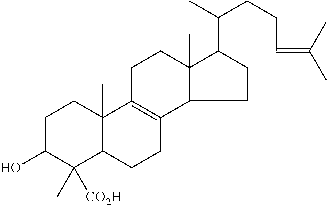 [SMILES: CC(C)=CCCC(C)C1CCC2C3=C(CCC21C)C1(C)CCC(O)C(C)(C(=O)O)C1CC3]

In some embodiments, agents useful in the methods described herein can be identified by screening compound libraries (including sterol libraries) to identify compounds that induce Rorγ activity (e.g., in a reporter assay, such as those described in Hu et al., Nature Chemical Biology 11:141-147 (2015), Soroosh et al., Proc. Natl. Acad. Sci. USA 111:12163-12168 (2014) and Santori et al., Cell Metabolism 21:286-297 (2015) and U.S. Pat. No. 8,389,739, each of which is hereby incorporated by reference).

Agents useful in the methods disclosed herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994)J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

### Pharmaceutical Compositions

In certain embodiments, provided herein is a composition, e.g., a pharmaceutical composition, containing at least one agent described herein together with a pharmaceutically acceptable carrier. In one embodiment, the composition includes a combination of multiple (e.g., two or more) agents described herein.

As described in detail below, the pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

### Methods

In certain aspects, provided herein are methods for inducing production of Rorγ⁺Helios⁻ LP Tregs and/or for treating or preventing a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis).

The methods described herein can be used to treat any subject in need thereof. As used herein, a “subject in need thereof” includes any subject that has a disease or disorder associated with a pathological immune response (e.g., an inflammatory bowel disease), as well as any subject with an increased likelihood of acquiring a such a disease or disorder.

The compositions described herein can be used, for example, as a composition for preventing or treating (reducing, partially or completely, the adverse effects of) an autoimmune disease. such as chronic inflammatory bowel disease, systemic lupus erythematosus, rheumatoid arthritis, multiple sclerosis, or Hashimoto's disease; an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a supplement, food, or beverage for improving immune functions; or a reagent for suppressing the proliferation or function of effector T-cells.

In some embodiments, provided herein are methods of treating an inflammatory bowel disease. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

In some embodiments, administration is in combination with administration of at least one prebiotic substance (e.g., a prebiotic substance that favors the growth of the bacterial species in the composition over the growth of other human commensal bacterial species). In some embodiments, the prebiotic substance is a nondigestible oligosaccharide. In some embodiments, the prebiotic substance is almond skin, inulin, oligofructose, raffinose, lactulose, pectin, hemicellulose, amylopectin, acetyl-Co A, biotin, beet molasses, yeast extracts, and resistant starch.

In some embodiments, the compositions described herein are administered in combination with an immunosuppressive agent. Examples of immunosuppressive agents include corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, anti-TNF inhibitors such as infliximab, adalimumab, certolizumab pegol, golimumab, or etanercept, and combinations thereof. Also described herein is a composition that comprises the bacterial composition and at least one substance selected from the group consisting of corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, anti-TNF inhibitors such as infliximab, adalimumab, certolizumab pegol, golimumab, or etanercept, and combinations thereof.

In some embodiments, the methods provided herein include the step of administering at least one antibiotic before or in combination with, the administration of a composition described herein.

In some embodiments, the methods provided herein include the step of determining the subject's microbiome prior to the administration of a composition described herein. In some embodiments, the selection of the bacteria or combination of bacteria administered to the subject is determined based upon the make-up of the subject's microbiome.

Actual dosage levels of the bacteria in the compositions described herein may be varied so as to obtain an amount of the bacteria which is effective to achieve the desired therapeutic response for a particular patient. A physician or veterinarian having ordinary skill in the art can readily determine and prescribe the effective amount of the composition required.

The pharmaceutical compositions disclosed herein may be delivered by any suitable route of administration, including orally and parenterally. In certain embodiments the pharmaceutical compositions are delivered generally (e.g., via oral or parenteral administration).

### EXEMPLIFICATION

### Experimental Procedures

### Mice

Mice were bred in a pathogen-free facility or in germ-free isolators. For IL-33 administration, recombinant mouse IL-33 (BioLegend) was administered i.p. at 2 μg/injection every other day for 7 days.

### Preparation of Lymphocytes and Flow Cytometry

Intestinal tissues were treated with RPMI containing 1 mM dithiothreitol, 20 mM EDTA and 2% FBS at 37 C for 15 min to remove epithelial cells. The tissues were then minced and dissociated in collagenase solution containing 1.5 mg/ml collagenase II (Gibco) 0.5 mg/ml, dispase and 1% FBS containing RPMI solution constantly stirring at 37° C. for 45 min. Single cell suspensions were then filtered and washed with 4% RPMI solution. The Peyer's Patches were treated in a similar fashion except for the first step of removal of epithelial cells. Lymph nodes and spleens were mechanically disrupted.

Single-cell suspensions were stained with antibodies against CD4, CD8, TCR-b, CD45, IL-17A, IFNγ, Helios (Biolegend), Rorγ, Foxp3, ST2 ((eBioscience), Nrp1 (R&D Systems), anti-ST2 conjugated to biotin (mdBioproducts). For cytokine analysis, cells were treated with RMPI containing 10% FBS, 10 ng/ml phorbol 12-myristate 13-acetate (Sigma), 1 μM Ionomycin (Sigma) in presence of GolgiStop (BD Biosciences) for 3.5 hours. For intracellular staining of cytokines and transcription factors, cells were stained for surface markers and fixed in eBioscience Fix/Perm buffer overnight, followed by permeabilization in eBioscience permeabilization buffer for 45 min in the presence of antibodies. Cells were acquired with a BD LSRII and analysis was performed with FlowJo (Tree Star) software.

### Gene Expression Profiling

Cells were double-sorted into TRIzol (Invitrogen) using a MoFlo sorter. All samples were generated in duplicate or triplicate. Sample processing and data analysis were performed on GeneChip Mouse Genome M1.0 ST chip arrays (Affymetrix) as described in D. Cipolletta et al., Nature 486, 549 (2012), which is hereby incorporated by reference.

### TNBS Colitis

Mice were desensitized via administration of 1% TNBS (prepared in 4:1 acetone:olive oil solution) on shaven skin between the shoulders. A week later, colitis was induced by intrarectal administration of 150-200 μg TNBS per gram of mouse (Sigma) in 50% ethanol into anaesthetized mice via a thin round-tip needle. The TNBS concentration was optimized for each batch of TNBS used in both germ free and SPF mice. The tip of the needle was inserted 4 cm proximal to the anal verge, and mice were held in a vertical position for at least 1 min after the injection. All the mice were observed and weighed daily and were sacrificed on day 4 after intrarectal TNBS administration at the peak of the disease. A combined colitis score was calculated based on weight loss, histology, diameter of the colon, and the appearance of the stool. For TNBS induction in gnobiotic mice, mice were colonized with single bacterial species for 2 weeks, as described earlier, prior to TNBS administration. Weight loss was scored as follows: 0, 0-4% weight loss or weight gain; 1, 4-10% weight loss; 2, 10-15% weight loss; 3, 15-20% weight loss; 4, more than 20% weight loss. The appearance of the stool was assessed as follows: 0, hard; 1, softened stool; 2, diarrhea; 3, bloody diarrhea. The thickness of the colon was assessed based on differential in diameter (Δdiameter=diameter of TNBS treated colon−diameter of untreated colon): 0, no change; 1, Δdiameter<1 mm; 2, Δdiameter=1-2 mm; 3, Δdiameter=2-3 mm; 4, Δdiameter>3 mm.

### Histology

Colons were fixed with Bouin's fixative, sectioned, and stained with haematoxylin and eosin. The degree of inflammation in the distal part of colon was graded from 0 to 4 as follows: 0, normal intact structure; 1, mild inflammation with intact structure; 2, infiltration of leukocytes and some damage to structure; 3, severe inflammation accompanied by complete loss of structure; 4, necrosis of the tissue.

### Bioinformatic and Statistical Analysis

Significance was assessed by the Student's t test. When necessary paired t test was used and a p value of <0.05 was deemed statistically significant.

Microarray data were background-corrected and normalized using the robust multi-array average (RMA) algorithm implemented in the GenePattern software package and replicates were averaged. Genes that have a mean expression value of at least 120 in any of the samples with a coefficient of variance set for >0.25 were analyzed. Genes that over- or under-represented in colonic Tregs compared with splenic Tregs or in Rorγ⁺ Tregs compared with Rorγ⁻ Tregs were determined by at least 2-fold or 1.5-fold differential expression respectively. Colon-preferential and tissue-specific gene clusters were determined by clustering genes that are over or under-expressed by 2-fold or higher in any of the tissues compared with spleen. Hierarchical clustering analysis used Pearson correlation in all comparisons. Genes that were over- or under-represented by 1.5 fold or higher in colonic Tregs compared with all non-lymphoid tissues were considered colon-preferential. Genes that have p values>0.05 at and FDR of 0.1 were deemed significant.

### Example 1: Expression of Rorγ by Colonic Regulatory T Cells

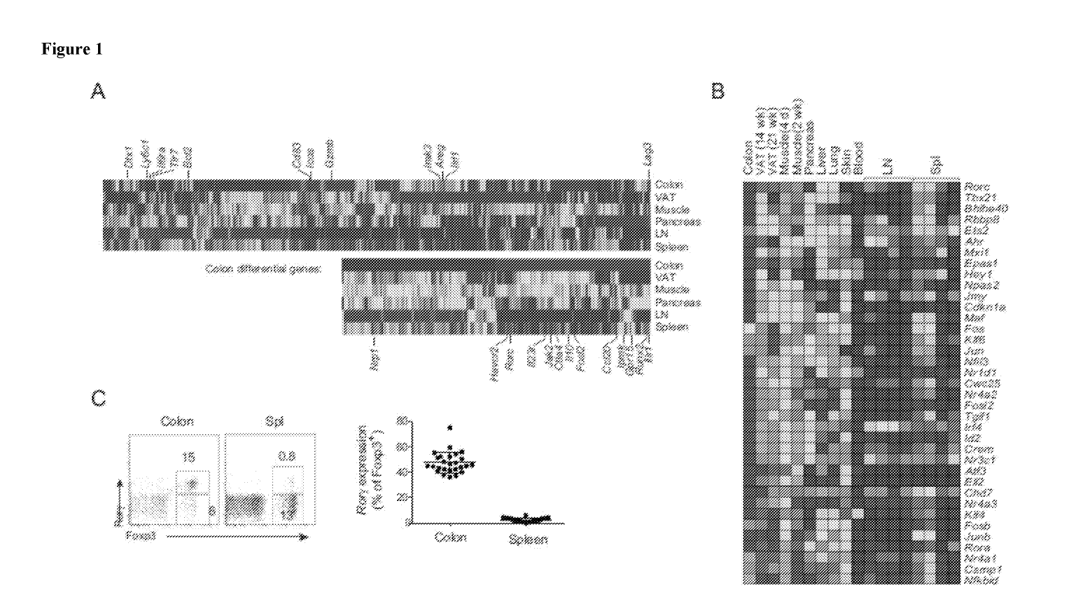

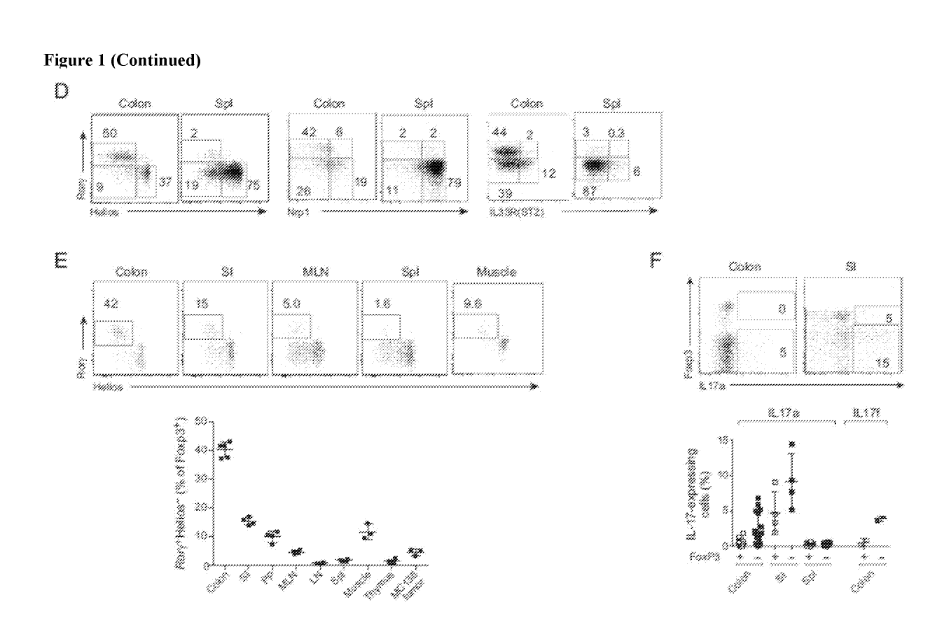

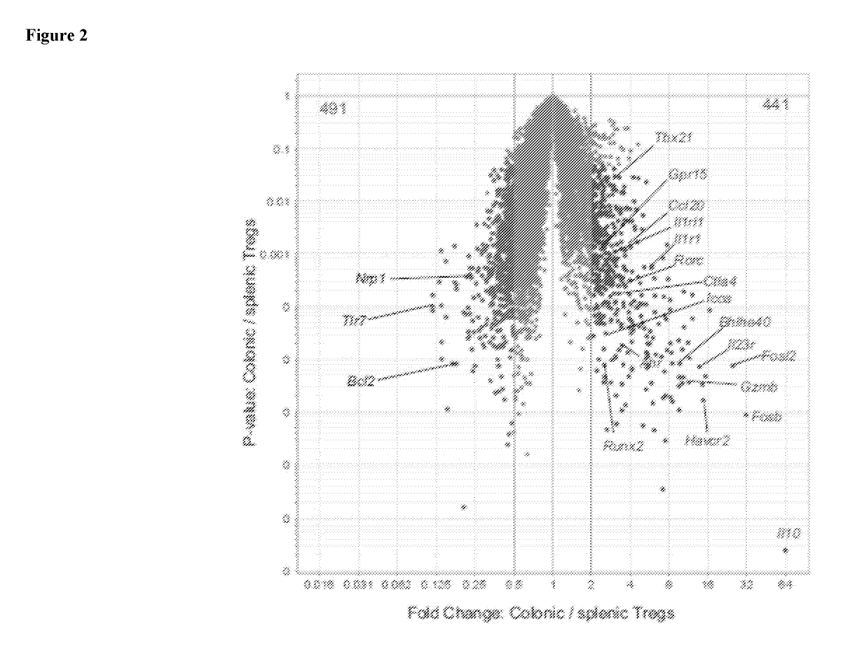

Comparison of gene expression profiles of highly purified CD4⁺FoxP3⁺ Tregs (from Foxp3^{igfp} reporter mice, described Bettelli et al., Nature 441, 235 (2006), which is hereby incorporated by reference) from colon or spleen uncovered 933 differential transcripts (at a FoldChange>2 and FDR<0.1; FIG. 1A (top), FIG. 2). Among these differential transcripts, several important signaling and effector pathways (including Icos, Gzmb, Lag3, Areg, IL1r11; FIG. 1A (top)), were shared in patchwork manner by other tissue Tregs, but ˜39% (at a colon specific bias >1.5 fold compared to other tissues) had preferential expression in colonic Tregs (including 1110, Ctla4, Havcr2, Ccl20, Jak2, Fosl2; FIG. 1A (bottom)). Correspondingly, several transcription factors were over-expressed in colon, including Ahr, Epas1, Hey1, Bcl6, Npas2, Nr1d1, and Maf. The most differential of these transcription factors was Rorc (gene that encodes Rorγ; FIG. 1B, 1C). Rorγ controls many aspects of innate and adaptive immunocyte differentiation, and is known as the key regulator of IL17 producing CD4⁺ T cells (Th17), and as a reciprocal antagonist of FoxP3 during in vitro differentiation in which iTreg and Th17 represent alternative cell fates (reviewed in Korn et al., Annu. Rev. Immunol 27, 485 (2009), which is hereby incorporated by reference).

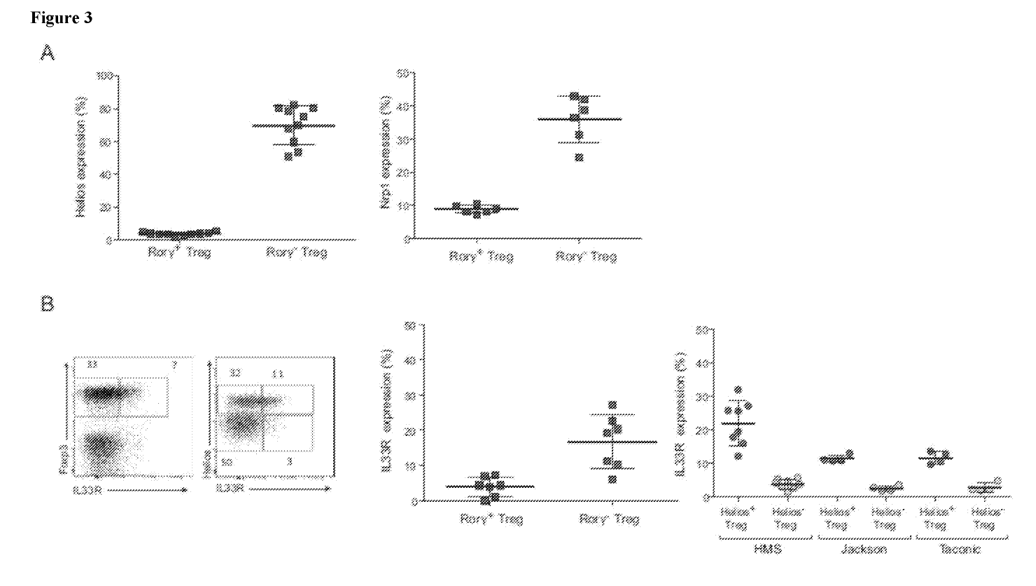

Flow cytometry confirmed that a large proportion of colonic CD4ToxP3⁺ Tregs indeed expressed Rorγ (40-60% in adult SPF mice), a phenotype largely absent in spleen or lymph node (LN) (FIG. 1C). Helios and Nrp1, which are considered markers of thymus-derived Tregs (reviewed in Bilate and Lafaille, Annu. Rev. Immunol. 30, 733 (2012), which is hereby incorporated by reference), were absent on colonic Rorγ⁺ Tregs (FIG. 1D, FIG. 3A). The combination of Rorγ and Helios demarcated three distinct subsets of colonic Tregs, Rorγ⁺ representing the majority of Helios⁻ cells (FIG. 1D, 3A left). This pattern was also seen for Treg cells in other tissues, and consistent with the RNA data. Rorγ⁺ Tregs were also detected in the small intestine and in the regenerating muscle, albeit at lower frequencies (FIG. 1E). Rorγ⁺ Tregs were distinct from those expressing the IL-33 receptor, most of which were Helios⁺ (FIG. 1D, FIG. 3B). Rare Tregs expressing IL-17 and Rorγ have been observed during chronic inflammation or cancer, usually with high levels of Helios suggestive of a thymic origin (reviewed in Du et al., J. Leukoc. Biol. 96, 39 (2014), hereby incorporated by reference). IL-17 production in colonic Rorγ⁺ Tregs was tested. In contrast to the clear subset of IL-17 expressing Tregs in the small intestine LP, colonic Rorγ⁺ Tregs did not secrete IL-17A or F (FIG. 1F).

### Example 2: Certain Species of Gut Microbacteria Induce Rorγ⁺ Regulatory T Cells

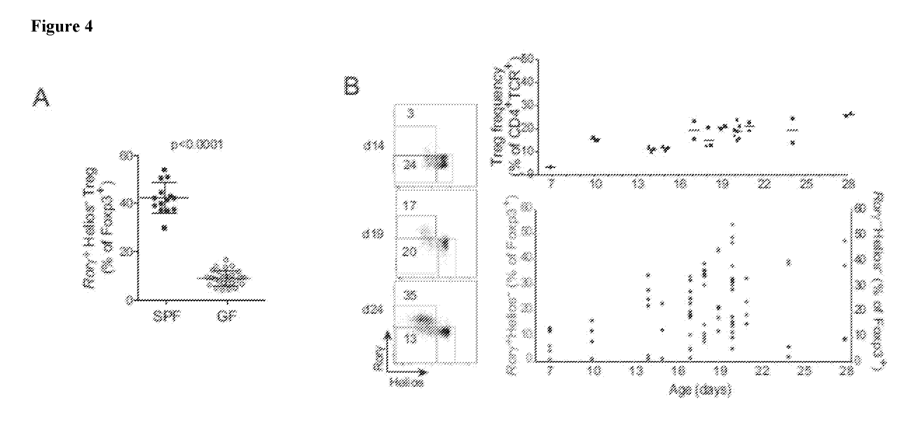

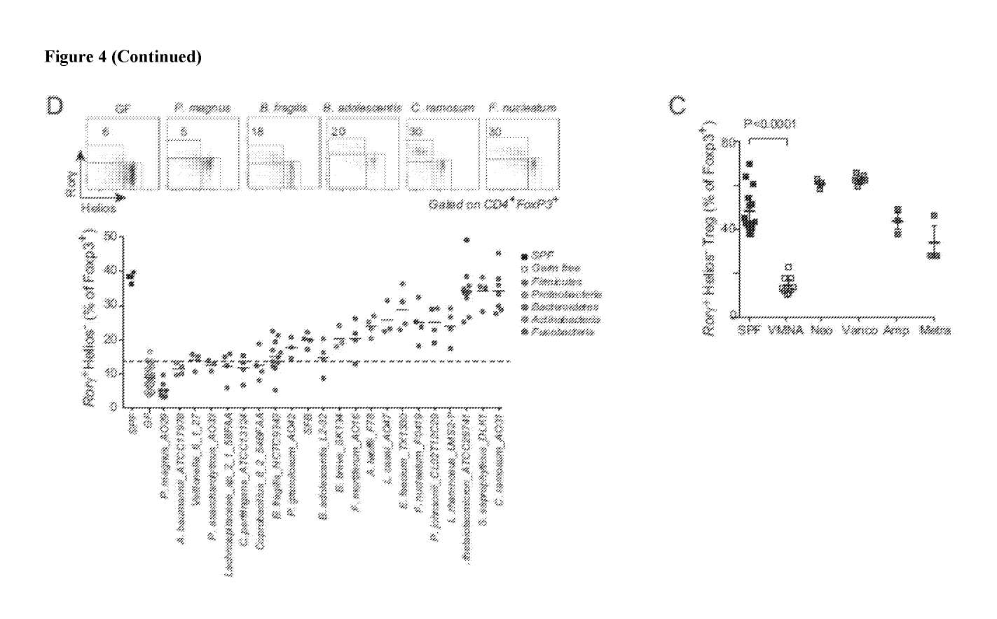

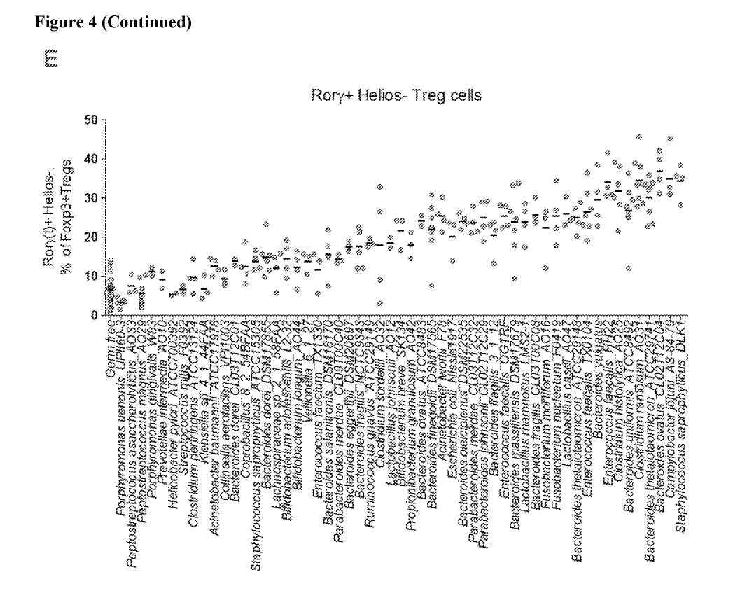

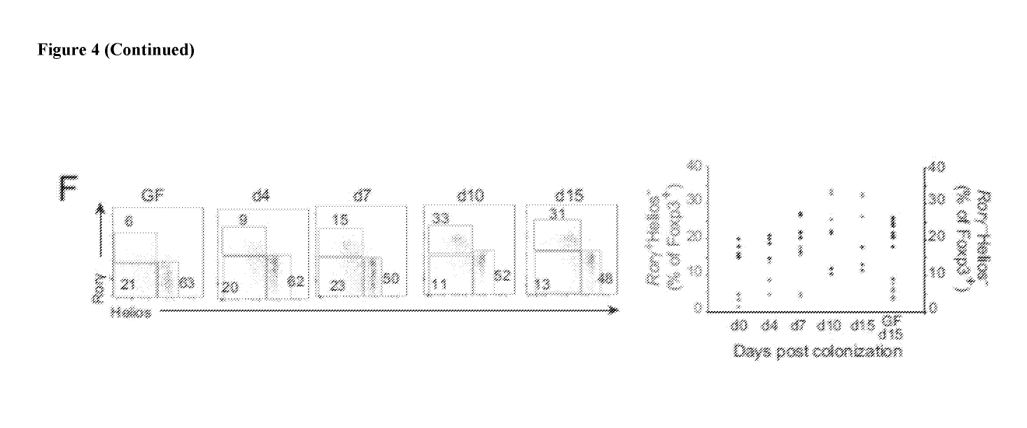

The properties of colonic Rorγ⁺ Treg population suggested a link to the gut microbiota. Germ free (GF) mice had a far lower proportion of Rorγ⁺ Tregs than their conventionally-raised specific-pathogen-free (SPF) counterparts (FIG. 4A). During normal maturation of SPF mice, Rorγ⁺ Tregs appeared between 15 and 25 days of age (FIG. 4B), coincident with the radical changes in the gut microbiota that accompany the transition to solid food. Interestingly, Rorγ⁺ Tregs appeared a few days after cells with a Rorγ⁻Helios phenotype. Oral antibiotic treatment of adult mice strongly affected Rorγ⁺ Tregs (FIG. 4C), a large reduction following a broad-spectrum combination (vancomycin, neomycin, metronidazole, ampicillin “VMNA”) while individual antibiotics have less or no effect suggested the contribution of several microbes.

A panel of bacterial species selected from the microbiota of the human gastrointestinal tract were tested for their ability to induce the production of Rorγ⁺ Tregs. Bacteria were obtained from the ATCC, BEI Resources, or the German Collection of Microorganisms and Cell Cultures (DSMZ), or from the laboratory collections at Harvard Medical School. Anaerobic bacteria were cultured under strictly anaerobic conditions (80% N₂, 10% H₂, 10% CO₂) at 37° C. in an anaerobic chamber.

Germfree C57BL/6J were maintained in sterile isolators, and were inoculated by gavage with single bacterial species at 4 weeks of age, then housed in gnobiotic isolators. After 2 weeks, mice were sacrificed, colon tissue was harvested, cleaned of luminal content and treated with RPMI containing 1 mM DTT, 20 mM EDTA and 2% FBS at 37° C. for 15 min to remove epithelial cells. The colon tissue was then minced and dissociated in collagenase solution (1.5 mg/ml collagenase II (Gibco), 0.5 mg/ml dispase and 1% FBS in RPMI) with constantly stirring at 37° C. for 45 min. Single cell suspensions were then filtered and washed in RPMI buffer, and stained with antibodies against CD4, CD8, TCR-13, CD45, then fixed in eBioscience Fix/Perm buffer overnight, followed by permeabilization in eBioscience permeabilization buffer for 45 min in the presence of conjugated anti-Foxp3 and anti-Rorγ antibodies. Cells were analyzed with a BD LSRII flow cytometer, and data analysis was performed with FlowJo (Tree Star) software. The proportion of Rorγ⁺ Tregs was determined within the total population of CD4⁺FoxP3⁺ Treg cells, and the significance of the difference relative to unmanipulated germfree mice determined using a two-tailed Student's T-test. False-discovery rates were estimated using the Benjamini-Hochberg procedure.

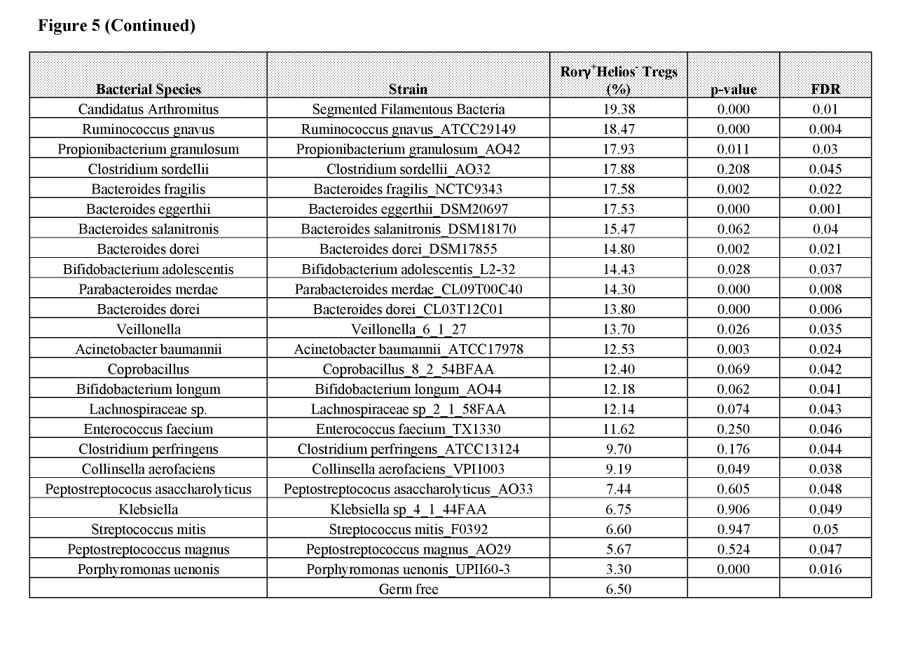

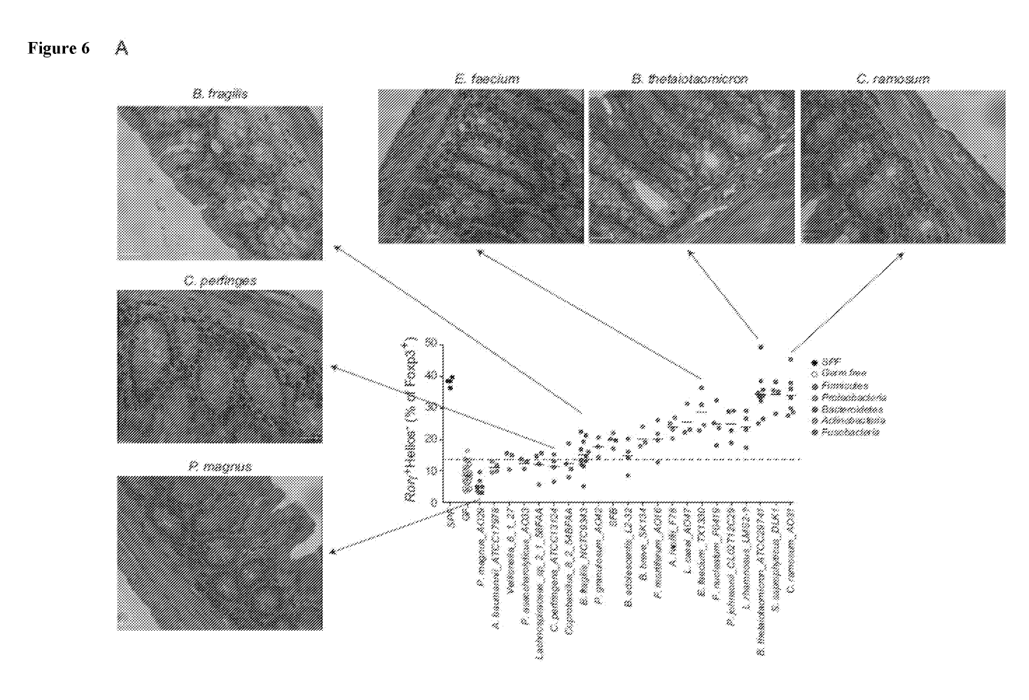

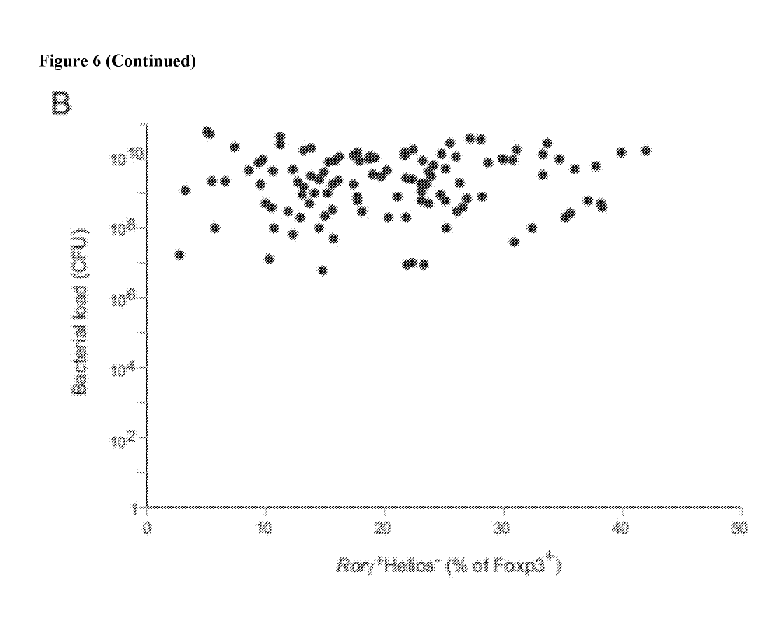

A number of microbes elicited colonic Rorγ⁺ Tregs, with a reproducible gradient of responses, and for some at frequencies comparable with those of SPF mice (FIGS. 4D and 5). This restoration of Rorγ⁺ Tregs was independent of bacterial load and not accompanied by inflammation (FIG. 6). Bacteria able to induce Rorγ⁺ Treg (and FoxP3⁺ Tregs more generally) belonged to several phyla and genera. The ability to promote colonic Treg production within the Bacteroides genus varied (high proportions with B. thetaiotaomicron, low with B. fragilis). A wider Bacteroides panel was assessed (FIGS. 4E and 5). Here again, a range of abilities to induce colonic Rorγ⁺ Tregs was observed. Colonic Rorγ⁺ Tregs did not appear immediately after GF colonization, but only after a few days. Here again, Rorγ⁺Helios⁻ Tregs appeared to follow Rorγ⁻Helios⁻ cells (FIG. 4F), suggesting that the Rorγ Helios⁻ cells are a first differentiation intermediate, prior to the induction of Rorγ.

### Example 3: Reduced Gut Regulatory T Cells in Rorγ Deficient Mice

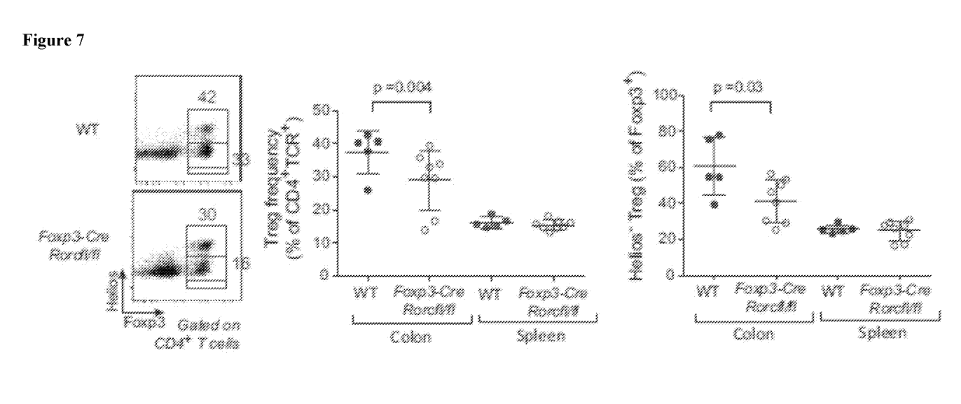

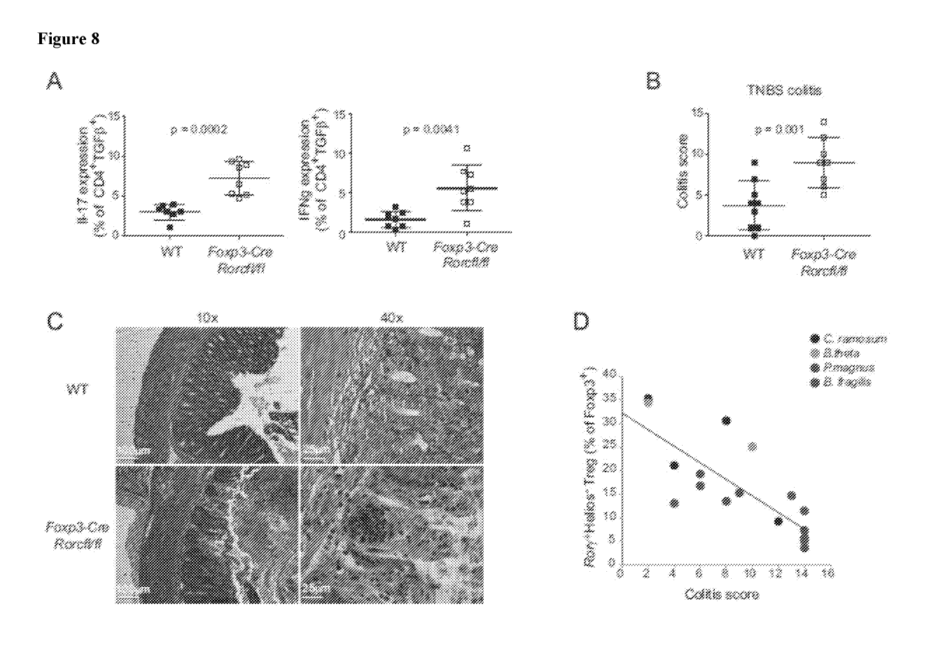

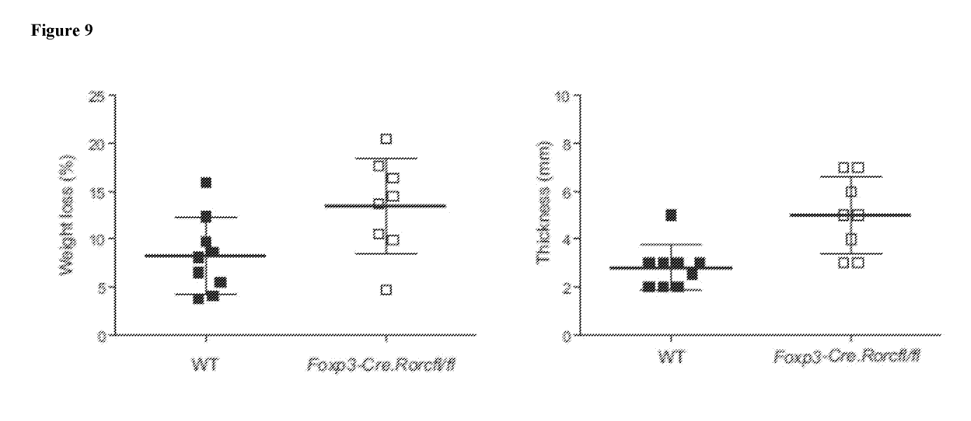

Foxp3-cre Rorc^{fl/fl} mice were generated. Such mice have a Treg-selective deficiency in Rorc. These mice do not show systemic Treg deficiency or scurfy-like pathology, but the amount of total colonic Tregs, and more specifically that of Helios⁻ Tregs, was reduced (FIG. 7). The loss of Rorγ⁺ Tregs in Foxp3-cre Rorc^{fl/fl} mice was accompanied by an increase of IL-17 and IFNγ production among FoxP3⁻ conventional CD4⁺ T cells (FIG. 8A), suggesting a decreased regulatory activity of colonic Tregs in the absence of Rorγ. To verify this point, Foxp3-cre Rorc^{fl/fl} mice were assessed in the Trinitrobenzenesulfonic acid-(TNBS) induced colitis model. Foxp3-cre.Ror^{fl/fl} mice showed significant exacerbation of disease severity, reflected in the overall colitis score and the histopathology (FIGS. 8B and 8C, FIG. 9). In GF mice monocolonized with microbes that induce different levels of Rorγ⁺ Tregs, and challenged in the TNBS sensitization model, a significant correlation between the frequency of Rorγ⁺ Tregs and the colitis score was observed (FIG. 8D). These results demonstrate a non-redundant role for Rorγ⁺ Tregs in colonic homeostasis.

### Example 4: Rorγ Function in Rorγ⁻ Regulatory T Cells

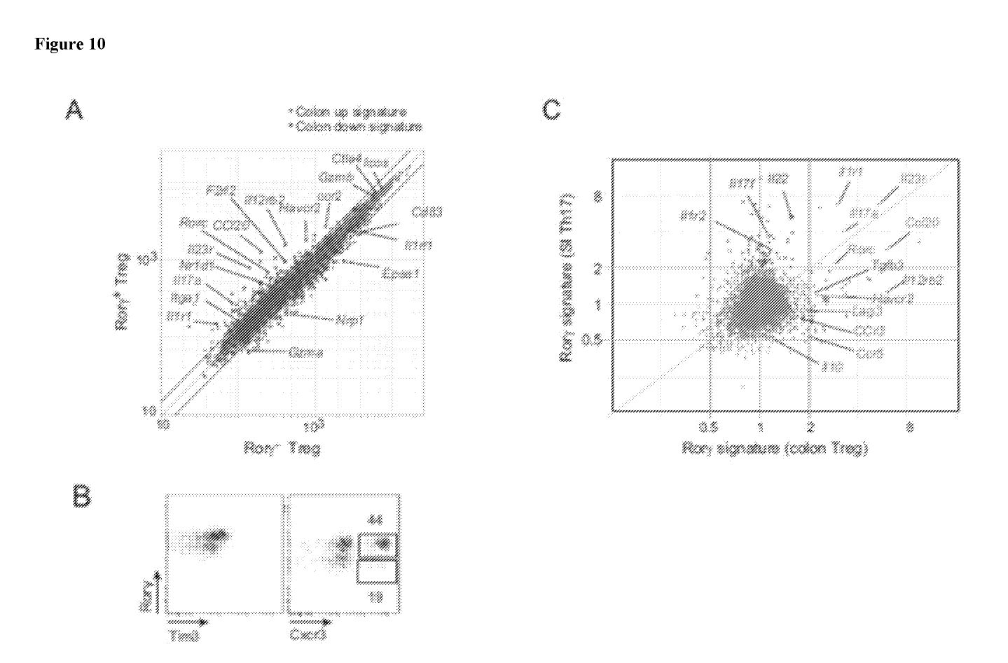

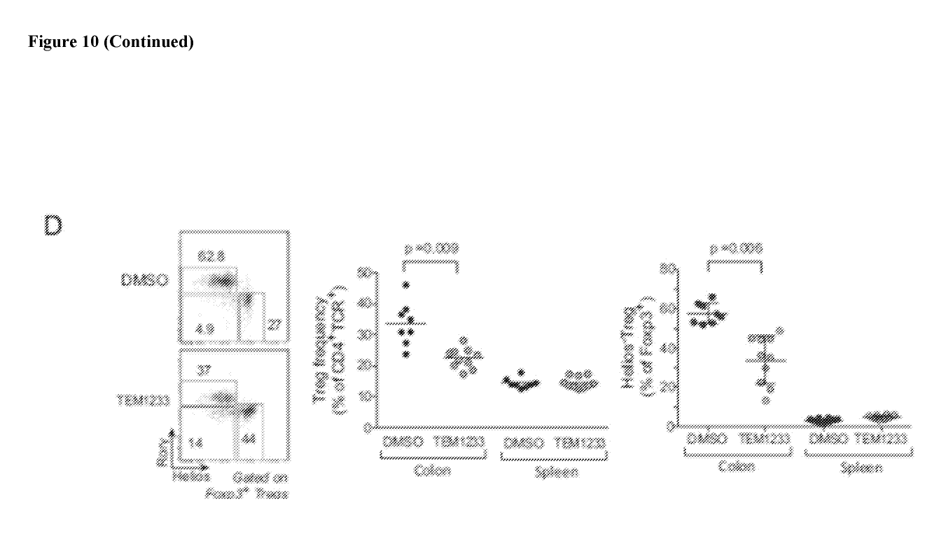

What transcripts Rorγ controls in Rorγ⁺ Tregs, and whether it is necessary to specify this particular Treg lineage was also examined. Gene expression profiles of Rorγ⁺ and Rorγ colonic Tregs (from an intercross of Foxp3^{thy 1.1} and Rorc^{gfp} reporter mice) were compared. Rorγ⁺ Tregs were enriched relative to Rorγ⁻ Tregs in some, but not all, transcripts of the colonic Treg signature (FIG. 10A), notably Il23r, Cxcr3, Tbx21 and Havcr2. Preferential expression of some of the corresponding proteins, including Cxcr3, was validated (FIG. 10B). Il1r11 (encodes IL-33R), Nrp1 and Ikzf2 were underrepresented in Rorγ⁺ Tregs.

It was next examined how the transcripts associated with Rorγ in colonic Tregs relate to the Rorγ-dependent signature in conventional Th17 cells, as defined from a comparison of CD4⁺ T cells from the small intestine of mice colonized, or not, with Segmented Filamentous Bacteria, classic inducers of Rorγ dependent Th17 cells (FIG. 10C). Much of the classic Th17 signature did not correlate with Rorγ in colonic Tregs (FIG. 10C), and there was only a minority of shared transcripts (e.g. Rorc itself, Il23r, Ccl20, as well as Il17a). In contrast, Rorγ in colonic Tregs was specifically associated with Treg preferential transcripts (FIG. 10C, e.g. Havrc2, Lag3, Tbx21).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of inducing the production of Rorγ⁺Helios⁻ lamina propria regulatory T cells in a subject comprising administering to the subject a composition comprising a bacteria selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

2. The method of claim 1, wherein the composition does not comprise a bacterium of the Clostridia class.

3. The method of claim 1 or claim 2, wherein at least 10% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

4. The method of claim 3, wherein at least 25% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

5. The method of claim 3, wherein at least 50% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

6. The method of claim 3, wherein at least 75% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

7. The method of claim 3, wherein at least 90% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

8. The method of claim 3, wherein at least 99% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

9. The method of any one of claims 1 to 8, wherein the bacteria is selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum and Parabacteroides johnsonii.

10. The method of any one of claims 1 to 8, wherein the bacteria is selected from the group consisting of Bacteroides ovatus, Campylobacter jejuni, Staphylococcus saprophyticus, Enterococcus faecalis, Bacteroides thetaiotaomicron and Bacteroides vulgatus.

11. The method of any one of claims 1 to 8, wherein the bacteria is selected from the group consisting of Bacteroides ovatus, Campylobacter jejuni, Staphylococcus saprophyticus and Enterococcus faecalis.

12. The method of any one of claims 1 to 8, wherein the bacteria is Bacteroides ovatus.

13. The method of any one of claims 1 to 8, wherein the bacteria is Campylobacter jejuni.

14. The method of any one of claims 1 to 8, wherein the bacteria is Staphylococcus saprophyticus.

15. The method of any one of claims 1 to 8, wherein the bacteria is Enterococcus faecalis.

16. The method of any one of claims 1 to 15, wherein the composition further comprises a pharmaceutically acceptable carrier.

17. The method of any one of claims 1 to 15, wherein the composition is a food product supplemented with the bacteria.

18. The method of any one of claims 1 to 17, wherein the subject has or is predisposed to a disease associated with a pathological immune response.

19. The method of any one of claim 1 or 18, wherein the subject has or is predisposed to an inflammatory bowel disease.

20. The method of claim 19, wherein the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease.

21. The method of claim 20, wherein the inflammatory bowel disease is Crohn's disease or ulcerative colitis.

22. The method of any one of claims 1 to 21, wherein the composition is administered orally.

23. The method of any one of claims 1 to 16 or 18 to 21, wherein the composition is administered rectally.

24. The method of any one of claims 1 to 23, wherein the bacteria are live, replication competent bacteria.

25. The method of any one of claims 1 to 24, wherein the subject had been administered an antibiotic less than a week prior to administration of the composition.

26. The method of claim 25, wherein the subject had been administered an antibiotic less than 3 days prior to administration of the composition.

27. The method of any one of claims 1 to 26, wherein the subject has reduced levels of commensal bacteria present in their gut.

28. A method of treating or preventing an inflammatory bowel disease in a subject comprising administering to the subject a composition comprising a bacteria selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

29. The method of claim 28, wherein the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease.

30. The method of claim 29, wherein the inflammatory bowel disease is Crohn's disease or ulcerative colitis.

31. The method of any one of claims 28 to 30, wherein the administration of the composition induces the production of Rorγ⁺Helios⁻ lamina propria regulatory T cells in the subject.

32. The method any one of claims 28 to 31, wherein the composition does not comprise a bacterium of the Clostridia class.

33. The method of any one of claims 28 to 32, wherein at least 10% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

34. The method of claim 33, wherein at least 25% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

35. The method of claim 33, wherein at least 50% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

36. The method of claim 33, wherein at least 75% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

37. The method of claim 33, wherein at least 90% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

38. The method of claim 33, wherein at least 99% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

39. The method of any one of claims 28 to 38, wherein the bacteria is selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum and Parabacteroides johnsonii.

40. The method of any one of claims 28 to 38, wherein the bacteria is selected from the group consisting of Bacteroides ovatus, Campylobacter jejuni, Staphylococcus saprophyticus, Enterococcus faecalis, Bacteroides thetaiotaomicron and Bacteroides vulgatus.

41. The method of any one of claims 28 to 38, wherein the bacteria is selected from the group consisting of Bacteroides ovatus, Campylobacter jejuni, Staphylococcus saprophyticus and Enterococcus faecalis.

42. The method of any one of claims 28 to 38, wherein the bacteria is Bacteroides ovatus.

43. The method of any one of claims 28 to 38, wherein the bacteria is Campylobacter jejuni.

44. The method of any one of claims 28 to 38, wherein the bacteria is Staphylococcus saprophyticus.

45. The method of any one of claims 28 to 38, wherein the bacteria is Enterococcus faecalis.

46. The method of any one of claims 28 to 45, wherein the composition further comprises a pharmaceutically acceptable carrier.

47. The method of any one of claims 28 to 45, wherein the composition is a food product supplemented with the bacteria.

48. The method of any one of claims 28 to 47, wherein the composition is administered orally.

49. The method of any one of claims 28 to 46, wherein the composition is administered rectally.

50. The method of any one of claims 28 to 49, wherein the bacteria are live, replication competent bacteria.

51. A composition for inducing the production of Rorγ⁺Helios⁻ lamina propria regulatory T cells in a subject comprising a bacteria selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fagilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

52. The composition of claim 51, wherein the composition does not comprise a bacterium of the Clostridia class.

53. The composition of claim 51 or claim 52, wherein at least 10% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

54. The composition of claim 53, wherein at least 25% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

55. The composition of claim 53, wherein at least 50% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

56. The composition of claim 53, wherein at least 75% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

57. The composition of claim 53, wherein at least 90% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

58. The composition of claim 53, wherein at least 99% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

59. The composition of any one of claims 51 to 58, wherein the bacteria is selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum and Parabacteroides johnsonii.

60. The composition of any one of claims 51 to 58, wherein the bacteria is selected from the group consisting of Bacteroides ovatus, Campylobacter jejuni, Staphylococcus saprophyticus, Enterococcus faecalis, Bacteroides thetaiotaomicron and Bacteroides vulgatus.

61. The composition of any one of claims 51 to 58, wherein the bacteria is selected from the group consisting of Bacteroides ovatus, Campylobacter jejuni, Staphylococcus saprophyticus and Enterococcus faecalis.

62. The composition of any one of claims 51 to 58, wherein the bacteria is Bacteroides ovatus.

63. The composition of any one of claims 51 to 58, wherein the bacteria is Campylobacter jejuni.

64. The composition of any one of claims 51 to 58, wherein the bacteria is Staphylococcus saprophyticus.

65. The composition of any one of claims 51 to 58, wherein the bacteria is Enterococcus faecalis.

66. The composition of any one of claims 51 to 65, wherein the composition further comprises a pharmaceutically acceptable carrier.

67. The composition of any one of claims 51 to 65, wherein the composition is a food product supplemented with the bacteria.

68. The composition of any one of claims 51 to 67, wherein the composition is formulated for oral administration.

69. The composition of any one of claims 51 to 66, wherein the composition is formulated for rectal administration.

70. The composition of any one of claims 51 to 69, wherein the bacteria are live, replication competent bacteria.

71. A method of making a composition for inducing the production of Rorγ⁺Helios⁻ lamina propria regulatory T cells in a subject comprising combining a bacteria selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve with a pharmaceutically acceptable carrier.

72. The method of claim 71, wherein the composition does not comprise a bacterium of the Clostridia class.

73. The method of claim 71 or claim 72, wherein at least 10% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

74. The method of claim 73, wherein at least 25% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

75. The method of claim 73, wherein at least 50% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

76. The method of claim 73, wherein at least 75% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

77. The method of claim 73, wherein at least 90% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

78. The method of claim 73, wherein at least 99% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

79. The method of any one of claims 71 to 78, wherein the bacteria is selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum and Parabacteroides johnsonii.

80. The method of any one of claims 71 to 78, wherein the bacteria is selected from the group consisting of Bacteroides ovatus, Campylobacter jejuni, Staphylococcus saprophyticus, Enterococcus faecalis, Bacteroides thetaiotaomicron and Bacteroides vulgatus.

81. The method of any one of claims 71 to 78, wherein the bacteria is selected from the group consisting of Bacteroides ovatus, Campylobacter jejuni, Staphylococcus saprophyticus and Enterococcus faecalis.

82. The method of any one of claims 71 to 78, wherein the bacteria is Bacteroides ovatus.

83. The method of any one of claims 71 to 78, wherein the bacteria is Campylobacter jejuni.

84. The method of any one of claims 71 to 78, wherein the bacteria is Staphylococcus saprophyticus.

85. The method of any one of claims 71 to 78, wherein the bacteria is Enterococcus faecalis.

86. The method of any one of claims 71 to 85, wherein the composition is formulated for oral administration.

87. The method of any one of claims 71 to 85, wherein the composition is formulated for rectal administration.

88. The method of any one of claims 71 to 87, wherein the bacteria are live, replication competent bacteria.

89. A method of making a composition for inducing the production of Rorγ⁺Helios⁻ lamina propria regulatory T cells in a subject comprising combining a bacteria selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve with a food product.

90. The method of claim 89, wherein the composition does not comprise a bacterium of the Clostridia class.

91. The method of claim 89 or 90, wherein the food product comprises a dairy product.

92. The method of claim 91, wherein the dairy product is yogurt, frozen yogurt, ice cream, milk or cheese.

93. The method of claim 89 or 90, wherein the food product is a non-dairy food product.

94. The method of any one of claims 89 to 93, wherein the food product is a beverage.

95. The method of any one of claims 89 to 94, wherein at least 10% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fagilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

96. The method of claim 95, wherein at least 25% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

97. The method of claim 95, wherein at least 50% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

98. The method of claim 95, wherein at least 75% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

99. The method of claim 95, wherein at least 90% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

100. The method of claim 95, wherein at least 99% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

101. The method of any one of claims 89 to 100, wherein the bacteria is selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum and Parabacteroides johnsonii.

102. The method of any one of claims 89 to 100, wherein the bacteria is selected from the group consisting of Bacteroides ovatus, Campylobacter jejuni, Staphylococcus saprophyticus, Enterococcus faecalis, Bacteroides thetaiotaomicron and Bacteroides vulgatus.

103. The method of any one of claims 89 to 100, wherein the bacteria is selected from the group consisting of Bacteroides ovatus, Campylobacter jejuni, Staphylococcus saprophyticus and Enterococcus faecalis.

104. The method of any one of claims 89 to 100, wherein the bacteria is Bacteroides ovatus.

105. The method of any one of claims 89 to 100, wherein the bacteria is Campylobacter jejuni.

106. The method of any one of claims 89 to 100, wherein the bacteria is Staphylococcus saprophyticus.

107. The method of any one of claims 89 to 100, wherein the bacteria is Enterococcus faecalis.

108. The method of any one of claims 89 to 107, wherein the bacteria are live, replication competent bacteria.

109. A composition for inducing the production of Rorγ⁺Helios⁻ lamina propria regulatory T cells in a subject comprising a food product supplemented with a bacteria selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

110. The composition of claim 109, wherein the composition does not comprise a bacterium of the Clostridia class.

111. The composition of claim 109 or 110, wherein the food product comprises a dairy product.

112. The composition of claim 111, wherein the dairy product is yogurt, frozen yogurt, ice cream, milk or cheese.

113. The composition of claim 109 or 110, wherein the food product is a non-dairy food product.

114. The composition of any one of claims 109 to 113, wherein the food product is a beverage.

115. The composition of any one of claims 109 to 114, wherein at least 10% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fagilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

116. The composition of claim 115, wherein at least 25% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

117. The composition of claim 115, wherein at least 50% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

118. The composition of claim 115, wherein at least 75% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

119. The composition of claim 115, wherein at least 90% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

120. The composition of claim 115, wherein at least 99% of the bacteria in the composition are selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

121. The composition of any one of claims 109 to 120, wherein the bacteria is selected from the group consisting of Bacteroides ovatus, Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum and Parabacteroides johnsonii.

122. The composition of any one of claims 109 to 120, wherein the bacteria is selected from the group consisting of Bacteroides ovatus, Campylobacter jejuni, Staphylococcus saprophyticus, Enterococcus faecalis, Bacteroides thetaiotaomicron and Bacteroides vulgatus.

123. The composition of any one of claims 109 to 120, wherein the bacteria is selected from the group consisting of Bacteroides ovatus, Campylobacter jejuni, Staphylococcus saprophyticus and Enterococcus faecalis.

124. The composition of any one of claims 109 to 120, wherein the bacteria is Bacteroides ovatus.

125. The composition of any one of claims 109 to 120, wherein the bacteria is Campylobacter jejuni.

126. The composition of any one of claims 109 to 120, wherein the bacteria is Staphylococcus saprophyticus.

127. The composition of any one of claims 109 to 120, wherein the bacteria is Enterococcus faecalis.

128. The composition of any one of claims 109 to 127, wherein the bacteria are live, replication competent bacteria.

129. The method or composition of any one of claims 1, 28, 51, 71, 89 or 109, wherein Bacteroides ovatus is present in combination with one or more of Campylobacter jejuni, Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

130. The method or composition of any one of claims 1, 28, 51, 71, 89, 109 and 129 wherein Campylobacter jejuni is present in combination with one or more of Staphylococcus saprophyticus, Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

131. The method or composition of any one of claims 1, 28, 51, 71, 89, 109, 129 and 130 wherein Staphylococcus saprophyticus is present in combination with one or more of Enterococcus faecalis, Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

132. The method or composition of any one of claims 1, 28, 51, 71, 89, 109 and 129 to 131, wherein Enterococcus faecalis is present in combination with one or more of Bacteroides thetaiotaomicron, Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

133. The method or composition of any one of claims 1, 28, 51, 71, 89, 109 and 129 to 132, wherein Bacteroides thetaiotaomicron is present in combination with one or more Bacteroides vulgatus, Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

134. The method or composition of any one of claims 1, 28, 51, 71, 89, 109 and 129 to 133, wherein Bacteroides vulgatus is present in combination with one or more Bacteroides uniformis, Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

135. The method or composition of any one of claims 1, 28, 51, 71, 89, 109 and 129 to 134, wherein Bacteroides uniformis is present in combination with one or more Lactobacillus casei, Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

136. The method or composition of any one of claims 1, 28, 51, 71, 89, 109 and 129 to 135, wherein Lactobacillus casei is present in combination with one or more Bacteroides fragilis, Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

137. The method or composition of any one of claims 1, 28, 51, 71, 89, 109 and 129 to 136, wherein Bacteroides fragilis is present in combination with one or more Acinetobacter lwoffii, Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

138. The method or composition of any one of claims 1, 28, 51, 71, 89, 109 and 129 to 137, wherein Acinetobacter lwoffii is present in combination with one or more Fusobacterium nucleatum, Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

139. The method or composition of any one of claims 1, 28, 51, 71, 89, 109 and 129 to 138, wherein Fusobacterium nucleatum is present in combination with one or more Parabacteroides johnsonii, Bacteroides oleiciplenus, Lactobacillus rhamnosus, Bacteroides massiliensis, Parabacteroides merdae, Fusobacterium mortiferum, Bacteroides finegoldii and Bifidobacterium breve.

140. The method or composition of any one of claims 1, 28, 51, 71, 89, 109 and 129 to 139, wherein Parabacteroides johnsonii is present in combination with one or more Bacteroides oleiciplenus, Lactobacillus rhamnosus, Bacteroides massiliensis, Parabacteroides merdae, Fusobacterium mortiferum, Bacteroides finegoldii and Bifidobacterium breve.

141. The method or composition of any one of claims 1, 28, 51, 71, 89, 109 and 129 to 140, wherein Bacteroides oleiciplenus is present in combination with one or more Lactobacillus rhamnosus, Bacteroides massiliensis, Parabacteroides merdae, Fusobacterium mortiferum, Bacteroides finegoldii and Bifidobacterium breve.

142. The method or composition of any one of claims 1, 28, 51, 71, 89, 109 and 129 to 141, wherein Lactobacillus rhamnosus is present in combination with one or more Bacteroides massiliensis, Parabacteroides merdae, Fusobacterium mortiferum, Bacteroides finegoldii and Bifidobacterium breve.

143. The method or composition of any one of claims 1, 28, 51, 71, 89, 109 and 129 to 142, wherein Bacteroides massiliensis is present in combination with one or more Parabacteroides merdae, Fusobacterium mortiferum, Bacteroides finegoldii and Bifidobacterium breve.

144. The method or composition of any one of claims 1, 28, 51, 71, 89, 109 and 129 to 143, wherein Parabacteroides merdae is present in combination with one or more Fusobacterium mortiferum, Bacteroides finegoldii and Bifidobacterium breve.

145. The method or composition of any one of claims 1, 28, 51, 71, 89, 109 and 129 to 144, wherein Fusobacterium mortiferum is present in combination with one or more Bacteroides finegoldii and Bifidobacterium breve.

146. The method or composition of any one of claims 1, 28, 51, 71, 89, 109 and 129 to 145, wherein Bacteroides finegoldii is present in combination with Bifidobacterium breve.

147. The method or composition of any one of claims 129 to 146, wherein Clostridia ramosum is also present.

148. The method or composition of any one of claims 129 to 147, wherein Clostridia histolyticum is also present.

149. A method of inducing the production or activity of regulatory T cells in a subject comprising administering to the subject an agent that activates Rorγ.

150. The method of claim 149, wherein the regulatory T cells are lamina propria regulatory T cells.

151. The method of claim 149 or claim 150, wherein the subject has or is predisposed to a disease associated with a pathological immune response.

152. The method of claim 149 or 150, wherein the subject has or is predisposed to an inflammatory bowel disease.

153. The method of claim 152, wherein the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease.

154. The method of claim 152, wherein the inflammatory bowel disease is Crohn's disease or ulcerative colitis.

155. The method of any one of claims 149 to 154, wherein the agent is a small molecule.

156. The method of any one of claims 149 to 154, wherein the agent is a sterol.

157. The method of claim 156, wherein the sterol is selected from the group consisting of cholesterol sulfate, 25-OHC, 25-OHC sulfate, desmosterol, desmosterol sulfate, 5α,6α-epoxycholestanol sulfate, 7α,27-diOHC, 5α,6α-epoxycholestanol, 24S,25-epoxycholesterol, 7α-OHC, 20α-OHC, 22R-OHC, 24S-OHC, 27-OHC, 7β,27-OHC, 7-keto,27-OHC, 7α,27-OHC, 4AC4MΔ°, Δ7-daf, zymosterol, 4C, 7DHC, zymosterone, 4C22OH, 4,7-cholesten, 25OH, OR-12872 and OR-942.

158. The method of claim 156, wherein the sterol is 25-OHC, desmosterol or 7β,27-OHC.

159. A method of treating or preventing a disease associated with a pathological immune response in a subject comprising administering to the subject an agent that activates Rorγ.

160. The method of claim 159, wherein the disease associated with a pathological immune response is an inflammatory bowel disease.

161. The method of claim 160, wherein the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease.

162. The method of claim 161, wherein the inflammatory bowel disease is Crohn's disease or ulcerative colitis.

163. The method of any one of claims 149 to 162, wherein the agent is administered in an amount sufficient to induce the production or activity of regulatory T cells in the subject.

164. The method of claim 163, wherein the regulatory T cells are lamina propria regulatory T cells.

165. The method of any one of claims 159 to 164, wherein the agent is a small molecule.

166. The method of any one of claims 159 to 164, wherein the agent is a sterol.

167. The method of claim 166, wherein the sterol is selected from the group consisting of cholesterol sulfate, 25-OHC, 25-OHC sulfate, desmosterol, desmosterol sulfate, 5α,6α-epoxycholestanol sulfate, 7α,27-diOHC, 5α,6α-epoxycholestanol, 24S,25-epoxycholesterol, 7α-OHC, 20α-OHC, 22R-OHC, 24S-OHC, 27-OHC, 7β,27-OHC, 7-keto,27-OHC, 7α,27-OHC, 4AC4MΔ°, Δ7-daf, zymosterol, 4C, 7DHC, zymosterone, 4C22OH, 4,7-cholesten, 25OH, OR-12872 and OR-942.

168. The method of claim 166, wherein the sterol is 25-OHC, desmosterol or 7β,27-OH.
